(19) United States
(12) Patent Application Publication
(10) Pub. No.: US 2022/0305086 A1
(43) Pub. Date: Sep. 29, 2022

(54) METHOD FOR INCREASING LYMPHOCYTE COUNT BY USING IL-7 FUSION PROTEIN IN TUMORS

(71) Applicants: GENEXINE,I NC., Seongnam-si, Gyeonggi-do (KR); NEOIMMUNE TECH, INC., Rockville, MD (US)

(72) Inventors: Young Chul SUNG, Seoul (KR); Jung Won WOO, Seoul (KR); MinK yu HEO, Uiwang-si (KR); Sang In YANG, Yongin-si (KR); SeHwan YANG, Rockville, MD (US)

(73) Assignees: GENEXINE, INC., Seongnam-si, Gyeonggi-do (KR); NEOIMMUNE TECH, INC., Rockville, MD (US)

(21) Appl. No.: 17/638,516

(22) PCT Filed: Sep. 4, 2020
(86) PCT No.: PCT/US2020/049483

§ 371 Date: Feb. 25, 2022

Related U.S. Application Data
(60) Provisional application No. 62/895,787, filed on Sep. 4, 2019.
(60) Provisional application No. 62/935,828, filed on Nov. 15, 2019.

(51) Int. Cl. A61K 38/20; A61P 37/04; A61K 47/68

(52) U.S. Cl. CPC A61K 38/2046; A61P 37/04; A61K 47/6813

(57) ABSTRACT

A method for increasing a lymphocyte count in a subject in need thereof including administering to the subject (i) a modified interleukin-7 of the following formula (I): A−IL-7 wherein A is an oligopeptide consisting of 1 to 10 amino acid residues, and the IL-7 is a polypeptide which is capable of binding to IL-7 receptor; or (ii) an interleukin-7 fusion protein comprising (a) the modified interleukin-7, (b) a second domain containing an oligopeptide having 1 to 10 amino acid residues consisting of methionine, glycine, or a combination thereof; and (c) a third domain which prolongs the half-life of the interleukin-7 fusion protein.

### CROSS REFERENCE TO RELATED APPLICATIONS

This application claims benefits from U.S. Provisional Application No. 62/895,787 filed Sep. 4, 2019 and U.S. Provisional Application No. 62/935,828 filed Nov. 15, 2019.

### BACKGROUND

T cells recognize cancer antigens, destroy cancer cells, and differentiate and proliferate into memory cells, thus playing a pivotal role in the immune response to attack cancer. Therefore, diversity of recognition capable of recognizing various antigens expressed in T-cell cancer cells, expansion of T cell clones that respond to cancer specific antigens, and differentiation into memory T cells in cancer tissues can maximize the anticancer treatment. In addition, cytotoxic chemotherapy and radiation therapy, which are used as standard anticancer treatments, destroy bone marrow and immune cells in the blood, resulting in the loss of T cells essential for anticancer immune function, offsetting the anticancer effect of immunotherapy drugs. According to a recent report, deficiency of T cells in cancer patients is associated with a decrease in response rate to chemotherapy and a decrease in patient survival rate. To date, IL-2 (Aldesleukin®) is the only cytokine treatment that has been approved as a treatment that induces proliferation and hyperfunction of T cells. However, IL-2 administration has limited efficacy due to proliferation of regulatory T cells that induce immunosuppression, and serious side effects such as cytokine storm and capillary leak syndrome caused by excessive immune response limit actual clinical use of IL-2 administration.

In contrast, interleukin-7 (IL-7) is an important growth and activation factor for T cells, and is primarily involved in the differentiation, proliferation and survival of naïve T cells and memory T cells, which are involved in antigen recognition and targeting, while destroying cancer cells. It does not induce proliferation of regulatory T cells, which inhibits activating effector T cells. In addition, when the level of IL-7 increases in the body, T cell proliferation occurs, and through binding to IL-7 receptor (CD127), which is mainly expressed in the increased T cells, IL-7 moves into the cell (transcytosis). It maintains homeostasis and is known as the homeostatic cytokine. Therefore, severe immune-related side effects such as cytokine storm caused by excessive immune response, which have been a major problem in the clinical safety of other cytokine treatments, have not been reported in previous clinical trials with recombinant human IL-7 (rhIL-7) by Cytheris under codename CYT107.

IL-7 is encoded by the gene IL7 and binds to IL-7 receptor (CD127). IL-7 is an immunostimulatory cytokine which can promote immune responses mediated by B cells and T cells. IL-7 plays an important role in an adaptive immune system.

In copending application (U.S. application Ser. No. 15/126,313), of which the entire content is incorporated herein by reference, a modified IL-7 protein with enhanced expression and half-life, a fusion protein containing the modified IL-7, and their production method have been provided.

### SUMMARY

The present disclosure provides a method of increasing a lymphocyte count by employing the modified IL-7 or a fusion protein containing the modified IL-7.

An embodiment provides a method of increasing a lymphocyte count in a subject in need thereof, comprising administering the modified IL-7 or its fusion protein to the subject.

The subject in need thereof may be a mammal suffering from a cancer or a malignant tumor. The cancer may be a solid tumor. In an exemplary embodiment, the solid tumor may be locally advanced or metastatic solid tumors or glioblastoma.

In an embodiment, the subject may be human.

In an embodiment, the subject may previously or concurrently undergo a cancer treatment. The modified IL-7 or its fusion protein may be administered separately or simultaneously with another anti-cancer drug(s).

Therefore, various aspects of the disclosure include the following embodiments.

1. A method for increasing a lymphocyte count in a subject in need thereof, comprising administering

(i) a modified interleukin-7 of the following formula (I):

A−IL-7 formula (I)
wherein A is an oligopeptide consisting of 1 to 10 amino acid residues, and
the IL-7 is a polypeptide which is capable of binding to IL-7 receptor; or

(ii) an interleukin-7 fusion protein comprising
a first domain comprising the modified interleukin-7 of formula (I),
a second domain comprising an oligopeptide having 1 to 10 amino acid residues consisting of methionine, glycine, or a combination thereof; and
a third domain which prolongs the half-life of the interleukin-7 fusion protein, to the subject at a dose of greater than 600 μg/kg.

2. The method of above 1, wherein the subject is suffering from a cancer, infection, chronic failure of the right ventricle of the heart, Hodgkin's disease and cancers of the lymphatic system, leukemia, a leak or rupture in the thoracic duct, side effects of prescription medications including anticancer agents (e.g., chemotherapy), antiviral agents, and glucocorticoids, malnutrition resulting from diets that are low in protein, radiation therapy, uremia, autoimmune disorders, immune deficiency syndromes, high stress levels, trauma, thymectomy, or a combination thereof, or idiopathic, acute radiation syndrome (ARS) or a combination thereof.

3. The method of above 1 or 2, the IL-7 has an amino acid sequence selected from the group consisting of SEQ ID NOS: 1 to 6.

4. The method of any one of above 1-3, wherein the A is linked to the N-terminal of the IL-7.

5. The method of any one of above 1-4, wherein A is methionine (M), glycine (G), methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, glycine-glycine-glycine, MMMM, MGMM, MGGM, MGGG, MGMG, GMMM, GMGG, GGGG, MMMMM, MMGMM, MMGGM, MGMMG, MMMMG, GGGGG, GGMMM, GGGMG, MGMGMG, MMMGGG, MMGGMM, GGMMGG, MGMGMGMG, MMMMGGGG, MMGGMMGG, MMMMGGGG, MGMGMGMGMG, or and MMMMMGGGGG.

6. The method of above 5, wherein the third domain is linked to the N-terminal or C-terminal of the first domain or the second domain. In an embodiment, the third omain is linked to the C-terminal of the second domain.

7. The method of any one of above 1-6, wherein the third domain is any one selected from the group consisting of an Fc region of immunoglobulin or a part thereof, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, and a combination thereof.

8. The method of any one of above 1-7, wherein the third domain comprises an Fc region of a modified immunoglobulin.

9. The method of any one of above 1-8, wherein the modified immunoglobulin is selected from the group consisting of IgG1, IgG2, IgG3, IgG4, IgA1, IgA2, IgD, IgE and a combination thereof.

10. The method of any one of above 1-9, wherein the Fc region of the modified immunoglobulin comprises a hinge region, a CH2 domain, and a CH3 domain from the N-terminal to the C-terminal direction,

wherein the hinge region comprises a human IgD hinge region,

the CH2 domain comprises a part of the amino acid residues of CH2 domain of human IgD and human IgG4, and

the CH3 domain comprises a part of the amino acid residues of the human IgG4 CH3 domain.

11. The method of any one of above 1-10, wherein the Fc region of the modified immunoglobulin is represented by the following Formula (I):

N′—(Z1)p-Y—Z2-Z3-Z4-C′ Formula (I)

wherein N′ is the N-terminal of a polypeptide and C′ is the C-terminal of a polypeptide;

p is an integer of 0 or 1;

Z1 is an amino acid sequence having 5 to 9 consecutive amino acid residues from the amino acid residue at position 98 toward the N-terminal, among the amino acid residues at positions from 90 to 98 of SEQ ID NO: 7;

Y is an amino acid sequence having 5 to 64 consecutive amino acid residues from the amino acid residue at position 162 toward the N-terminal, among the amino acid residues at positions from 99 to 162 of SEQ ID NO: 7;

Z2 is an amino acid sequence having 4 to 37 consecutive amino acid residues from the amino acid residue at position 163 toward the C-terminal, among the amino acid residues at positions from 163 to 199 of SEQ ID NO: 7;

Z3 is an amino acid sequence having 71 to 106 consecutive amino acid residues from the amino acid residue at position 220 toward the N-terminal, among the amino acid residues at positions from 115 to 220 of SEQ ID NO: 8; and

Z4 is an amino acid sequence having 80 to 107 consecutive amino acid residues from the amino acid residue at position 221 toward the C-terminal, among the amino acid residues at positions from 221 to 327 of SEQ ID NO: 8.

12. The method of any one of above 1-11, wherein the third domain has an amino acid sequence selected from the group consisting of SEQ ID NOS: 9 to 14.

13. The method of any one of above 2-12, wherein the cancer is a solid tumor, a cancer of lymphatic system, or leukemia.

14. The method of above 13, wherein the solid tumor is synovial sarcoma, infiltrating duct carcinoma, rectal cancer, colon cancer, ovary cancer, ascending colon cancer, anal cancer, invasive ductal carcinoma, adenocarcinoma, rectal cancer with paraaortic in metastatis, neuroendocrine carcinoma (cervix), sigmoid colon cancer, or glioblastoma.

15. The method of any one of above 1-14, wherein the subject has previously received or concurrently receives one or more of cancer treatments including surgery, radiation, and/or chemotherapy.

16. The method of any one of above 1-15, wherein the (i) modified interleukin-7 or the (ii) interleukin-7 fusion protein is administered at a dose in a range from greater than 600 μg/kg to about 2,000 μg/kg.

17. The method of any one of above 1-16, wherein the (i) modified interleukin-7 or the (ii) interleukin-7 fusion protein is administered at a dose of about 720 μg/kg or above, about 960 μg/kg or above, about 1,200 μg/kg or above, about 1,700 μg/kg or above, or about 2,000 μg/kg.

18. The method of any one of above 1-16, wherein the (i) modified interleukin-7 or the (ii) interleukin-7 fusion protein is administered at a dose of about 720 μg/kg or above, about 840 μg/kg or above, or about 1,440 μg/kg or above.

19. The method of any one of above 1-16, wherein the (i) modified interleukin-7 or the (ii) interleukin-7 fusion protein is administered at 720 μg/kg or above, 840 μg/kg or above, 960 μg/kg or above, 1,200 μg/kg or above, 1,440 μg/kg or above, 1,700 μg/kg or above, or 2,000 μg/kg.

20. The method of any one of above 1-19, wherein the (i) modified interleukin-7 or the (ii) interleukin-7 fusion protein is administered twice or more times at an interval of 1 week, 2 weeks, 3 weeks, 4 weeks, 5 weeks, 6 weeks, 7 weeks, 8 weeks, 9 weeks, 10 weeks, 11 weeks, or 12 weeks.

21. The method of any one of above 1-19, wherein the (i) modified interleukin-7 or the (ii) interleukin-7 fusion protein is administered twice or more times at an interval of 10 days, 20 days, 30 days, 40 days, 50 days, 60 days, 70 days, 80 days, 90 days, or 100 days.

22. The method of any one of above 1-21, wherein the (i) modified interleukin-7 or the (ii) interleukin-7 fusion protein is administered parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

23. The method of any one of above 1-22, comprising administering the (ii) interleukin-7 fusion protein.

24. The method of any one of above 1-23, wherein the (i) modified interleukin-7 has the amino acid sequence of SEQ ID NO: 18, and (ii) interleukin-7 fusion protein comprises the amino acid sequence of SEQ ID NO: 24.

25. The method of any one of above 1-24, wherein the subject has a lymphocyte count of 1000 lymphocyte cells or less/μl of blood and/or serum, as determined according to Common Terminology Criteria for Adverse Events (CTCAE) version 4.0.

26. The method of any one of above 1-25, wherein the lymphocyte is T-cell.

27. The method of any one of above 1-26, wherein the (i) modified interleukin-7 or the (ii) interleukin-7 fusion protein is administered twice or more times in an amount of about 720 μg/kg at an interval of about 1 week, about 2 weeks, about 3 weeks, or about 4 weeks.

28. The method of any one of above 1-26, wherein the (i) modified interleukin-7 or the (ii) interleukin-7 fusion protein is administered twice or more times in an amount of about 840 μg/kg at an interval of about 2 weeks, about 3 weeks, about 4 weeks, or about 5 weeks.

29. The method of any one of above 1-26, wherein the (i) modified interleukin-7 or the (ii) interleukin-7 fusion protein is administered twice or more times in an amount of about 960 μg/kg at an interval of about 2 weeks, about 3 weeks, about 4 weeks, about 5 weeks, or about 6 weeks.

30. The method of any one of above 1-26, wherein the (i) modified interleukin-7 or the (ii) interleukin-7 fusion protein is administered twice or more times in an amount of about 1200 μg/kg at an interval of about 3 weeks, about 4 weeks, about 5 weeks, about 6 weeks, about 7 weeks, or about 8 weeks.

31. The method of any one of above 1-26, wherein the (i) modified interleukin-7 or the (ii) interleukin-7 fusion protein is administered twice or more times in an amount of about 1440 μg/kg at an interval of about 3 weeks, about 4 weeks, about 5 weeks, about 6 weeks, about 7 weeks, about 8 weeks, about 10 weeks, or about 12 weeks, or about 2 months or 3 months.

32. The method of any one of above 1-26, wherein (i) modified interleukin-7 or the (ii) interleukin-7 fusion protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or about 2,000 μg/kg.

33. The method of any one of above 1-26, wherein the (i) modified interleukin-7 or the (ii) interleukin-7 fusion protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

34. The method of any one of above 1-26, wherein the (i) modified interleukin-7 or the (ii) interleukin-7 fusion protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

35. The method of any one of above 1-26, wherein the (i) modified interleukin-7 or the (ii) interleukin-7 fusion protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

36. The method of any one of above 32-35, wherein the (i) modified interleukin-7 or the (ii) interleukin-7 fusion protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

37. The method of above 26, wherein the T-cell is CD4⁺ T-cell and/or CD8⁺ T-cell.

38. The method of above 26, wherein the T-cell is CD4⁺/CD8⁺ T-cell.

39. The method of any one of above 1-38, wherein the subject has a lymphocyte count of 800 lymphocyte cells or less/μl of blood.

40. The method of any one of above 1-39, wherein the subject has a lymphocyte count of 500 lymphocyte cells or less/μl of blood.

41. The method of any one of above 1-40, wherein the subject has a lymphocyte count of 200 lymphocyte cells or less/μl of blood.

42. The method of any one of above 15-41, wherein the subject is concurrently administered with an anti-cancer agent.

43. The method of above 42, wherein the anti-cancer agent is an anti-cancer chemical compound.

44. The method of any one of above 1-43, wherein a number of tumor infiltrating lymphocytes (TILs) in the tumor is increased after the administration of the (i) modified interleukin-7 or the (ii) interleukin-7 fusion protein compared to a number of TILs in a tumor before administration.

45. The method of any one of above 1-44, wherein the TILs are CD4⁺ TILs.

46. The method of any one of above 1-44, wherein the TILs are CD8⁺ TILs.

47. The method of any one of above 44-46, wherein the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

48. The method of any one of above 1-26, wherein the (i) modified interleukin-7 or the (ii) interleukin-7 fusion protein is administered at a dose of about 650 μg/kg-680 μg/kg, about 680 μg/kg-700 μg/kg, about 700 μg/kg-720 μg/kg, about 720 μg/kg-740 μg/kg, about 740 μg/kg-750 μg/kg, about 750 μg/kg-760 μg/kg, about 760 μg/kg-780 μg/kg, about 780 μg/kg-800 μg/kg, about 800 μg/kg-820 μg/kg, about 820 μg/kg-840 μg/kg, about 840 μg/kg-850 μg/kg, about 850 μg/kg-860 μg/kg, about 860 μg/kg-880 μg/kg, about 880 μg/kg-900 μg/kg, about 900 μg/kg-920 μg/kg, about 920 μg/kg-940 μg/kg, about 940 μg/kg-950 μg/kg, about 950 μg/kg-960 μg/kg, about 960 μg/kg-980 μg/kg, about 980 μg/kg-1000 μg/kg, about 1,000 μg/kg-1,020 μg/kg, about 1,020 μg/kg-1,040 μg/kg, about 1,040 μg/kg-1,060 μg/kg, about 1,060 μg/kg-1,080 μg/kg, about 1,080 μg/kg-1,100 μg/kg, about 1,100 μg/kg-1,120 μg/kg, about 1,120 μg/kg-1,140 μg/kg, about 1,140 μg/kg-1,160 μg/kg, about 1,160 μg/kg-1,180 μg/kg, about 1,180 μg/kg-1,200 μg/kg, about 1,200 μg/kg-1,220 μg/kg, about 1,220 μg/kg-1,240 μg/kg, about 1,240 μg/kg-1,260 μg/kg, about 1,260 μg/kg-1,280 μg/kg, about 1,280 μg/kg-1,300 μg/kg, about 1,300 μg/kg-1,320 μg/kg, about 1,320 μg/kg-1,340 μg/kg, about 1,340 μg/kg-1,360 μg/kg, about 1,360 μg/kg-1,380 μg/kg, about 1,380 μg/kg-1,400 μg/kg, about 1,400 μg/kg-1,420 μg/kg, about 1,420 μg/kg-1,440 μg/kg, about 1,440 μg/kg-1,460 μg/kg, about 1,480 μg/kg, about 1,480 μg/kg-1,500 μg/kg, about 1,500 μg/kg-1,520 μg/kg, about 1,520 μg/kg-1,540 μg/kg, about 1,540 μg/kg-1,560 μg/kg, about 1,560 μg/kg-1,580 μg/kg, about 1,580 μg/kg-1,600 μg/kg, about 1,600 μg/kg-1,620 μg/kg, about 1,620 μg/kg-1,640 μg/kg, about 1,640 μg/kg-1,660 μg/kg, about 1,660 μg/kg-1,680 μg/kg, about 1,680 μg/kg-1,700 μg/kg, about 1,700 μg/kg-1,720 μg/kg, about 1,720 μg/kg-1,740 μg/kg, about 1,740 μg/kg-1,760 μg/kg, about 1,760 μg/kg-1,780 μg/kg, about 1,780 μg/kg-1,800 μg/kg, about 1,800 μg/kg-1,820 μg/kg, about 1,820 μg/kg-1,840 μg/kg, about 1,840 μg/kg-1,860 μg/kg, about 1,860 μg/kg-1,880 μg/kg, about 1,880 μg/kg-1,900 μg/kg, about 1,900 μg/kg-1,920 μg/kg, about 1,920 μg/kg-1,940 μg/kg, about 1,940 μg/kg-1,960 μg/kg, about 1,960 μg/kg-1,980 μg/kg, or about 1,980 μg/kg-2,000 μg/kg.

49. The method of above 48, wherein the (i) modified interleukin-7 or the (ii) interleukin-7 fusion protein is administered twice, three times, four times, or five times with the above dose at an interval of about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, or about 15 weeks.

### BRIEF DESCRIPTION OF DRAWINGS

FIGS. 1A, 1B, and 1C show pharmacokinetic profiles by IV, SC, and IM route in rats, respectively.

FIGS. 2A and 2B show GX-I7 concentration-time Profiles by Dose, shown in linear scale and log scale.

FIGS. 3A and 3B show serum exposure (Cₘₐₓ and AUCₗₐₛₜ) vs dose correlation, respectively.

FIGS. 4A and 4B show changes in absolute lymphocyte count (ALC) in comparison with the baseline value in low dose (60-120 μg/kg), medium dose (240-480 μg/kg), and high dose (720-1,200 μg/kg) groups. FIG. 4A shows the results from solid cancer patient group and FIG. 4B shows the results from glioblastoma patient group.

FIGS. 5A and 5B show changes in CD3+ counts in comparison with the baseline in low dose (60-120 μg/kg), medium dose (240-480 μg/kg), and high dose (720-1,200 μg/kg) groups. FIG. 5A shows the results from solid cancer patient group and FIG. 5B shows the results from glioblastoma patient group.

FIGS. 6A and 6B show changes in CD4⁺ counts in comparison with the baseline value in low dose (60-120 μg/kg), medium dose (240-480 μg/kg), and high dose (720-1,200 μg/kg) groups. FIG. 6A shows the results from solid cancer patient group and FIG. 6B shows the results from glioblastoma patient group.

FIGS. 7A and 7B show changes in CD8⁺ counts in comparison with the baseline value in low dose (60-120 μg/kg), medium dose (240-480 μg/kg), and high dose (720-1,200 μg/kg) groups. FIG. 7A shows the results from solid cancer patient group and FIG. 7B shows the results from glioblastoma patient group.

FIGS. 8A and 8B show changes in absolute lymphocyte count (ALC) in non-lymphopenia solid cancer patient group (baseline ALC≥1,000 cells/mm³) and lymphopenia (baseline ALC<1,000 cells/mm³) solid cancer patient group, respectively, who are divided into in low dose (60-120 μg/kg), medium dose (240-480 μg/kg), and high dose (720-1,200 μg/kg) groups. *p<0.05, **p<0.01, ***p<0.001 versus baseline (0 week) group by Wilcoxon matched-pairs signed rank test.

FIGS. 9A and 9B show changes in absolute lymphocyte count (ALC) in non-lymphopenia glioblastoma patient group (baseline ALC≥1,000 cells/mm³) and lymphopenia (baseline ALC<1,000 cells/mm³) solid cancer patient group, respectively, who are divided into in low dose (60-120 μg/kg), medium dose (240-480 μg/kg), and high dose (720-1,200 μg/kg) groups. *p<0.05, **p<0.01, ***p<0.001 versus baseline (0 week) group by Wilcoxon matched-pairs signed rank test.

FIGS. 10A, 10B, and 10C show changes in Ki67, CD127, and Treg ratio in CD4⁺ and CD8⁺ cells, respectively, after administration of IL-7 fusion protein in low dose (60-120 μg/kg), medium dose (240-480 μg/kg), and high dose (720-1,200 μg/kg) groups. NS: Not significant, *p<0.05, **p<0.01, ***p<0.001 versus baseline (0 week) group by Wilcoxon matched-pairs signed rank test.

FIG. 11 is an illustration of the timeline of blood sampling in monkey model.

FIGS. 12A and 12B show expression patterns of Ki67 in CD8⁺ and CD4⁺ cells, respectively, in blood samples of the tested monkeys.

FIGS. 13A and 13B show changes in absolute lymphocyte count (ALC) in comparison with the baseline value by multiple administrations over the time from time zero (0 week) to 15 weeks in low dose (120 μg/kg) group (FIG. 13A) and medium dose (360-600 μg/kg) and high dose (840-1,440 μg/kg) groups (FIG. 13B).

FIGS. 14A, 14B, and 14C show changes in absolute lymphocyte count (ALC) in comparison with the baseline value (week zero) by multiple administrations of IL-7 fusion protein at intervals of 8 weeks or longer in glioblastoma patents under chemotherapy using TMZ (Temozolomide 150 mg/m2), Avastin/Irinotecan (A; Avastin 10 mg/kg, I; Irinotecan 100 mg/m2), and PCV (PCV; CCNU 240 mg, Vincristine 2 mg, Procarbazine 150 mg&100 mg, Vincristine 2 mg), respectively.

FIGS. 15A and 15B show changes in the subsets of CD4⁺ and CD8⁺ T cells, and changes in chemokine receptor CCR and other immune cells (B cells and NK cells) after administration of IL-7 fusion protein. NS; Not significant, p>0.05, *p<0.05, **p<0.01, ***p<0.001 versus baseline (0 week) group by Wilcoxon matched-pairs signed rank test.

### EMBODIMENTS

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the aspects provided herein, because the scope of the aspects provided herein is limited only by the claims. Unless otherwise defined, all technical and scientific terms used herein have the same meaning as commonly understood by one of ordinary skill in the art to which aspects provided herein belong.

The term “pharmaceutical composition” is defined herein to refer to a mixture or solution containing at least one therapeutic agent to be administered to a mammal, in order to prevent or treat a particular disease or condition affecting the mammal. In embodiments, the mammal may be human.

The term “pharmaceutically acceptable” is defined herein to refer to those compounds, materials, compositions and/or dosage forms, which are, within the scope of sound medical judgment, suitable for contact with the tissues a human patient without excessive toxicity, irritation allergic response and other problem complications commensurate with a reasonable benefit/risk ratio.

The term “treating” or “treatment” as used herein comprises a treatment relieving, decrease, reducing or alleviating at least one symptom in a human patient or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “prevent”, “preventing” or “prevention” as used herein comprises the prevention of at least one symptom associated with or caused by the state, disease or disorder being prevented.

The term “pharmaceutically effective amount” or “clinically effective amount” of a combination of therapeutic agents is an amount sufficient to provide an observable improvement over the baseline clinically observable signs and symptoms of the disorder treated with the combination.

The phrase “a human patient in need of such treatment” as used herein refers to a human patient diagnosed with or suffering from the identified proliferative disease.

The term about” or “approximately” shall have the meaning of within 10%, 9%, 8%, 7%, 6%, or within 5%, of a given value or range.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

### Modified IL-7

A modified IL-7 that can be used in the embodiments may have the following structure:

A−IL-7 formula (I)

wherein A is an oligopeptide consisting of 1 to 10 amino acid residues, and IL-7 is an interleukin 7, a polypeptide capable of binding to IL-7 receptor (also known as CD127), or a polypeptide having the activity of IL-7 or a similar activity thereto.

As used herein, the term “a polypeptide having the activity of IL-7 or a similar activity thereof” refers to a polypeptide or protein having the same or similar sequence and activity to IL-7. Unless otherwise specified in an embodiment, the term can be used as a concept which is interchangeable with the first domain of the IL-7 fusion protein or the modified IL-7 fusion protein, as interchangeably used herein.

The IL-7 includes a polypeptide consisting of an amino acid sequence represented by SEQ ID NOS: 1 to 6. Additionally, IL-7 may have a sequence identity of about 70%, 75%, 80%, 85%, 86%, 87%, 88%, 89%, 90%, 91%, 92%, 93%, 94%, 95%, 96%, 97%, 98%, or 99% or higher, to the sequences of SEQ ID NOS: 1 to 6. The sequence identity of the peptide sequence may be determined by using a known sequence alignment or comparison software. For example, the sequence identity may be determined by using the BLASTP program (blast.ncbi.nlm.nih.gov/Blast.cgi?PAGE=Proteins) with its default setting.

The IL-7 may include an IL-7 protein or a fragment thereof, wherein the fragment is capable of binding to IL-7 receptor. As used herein, the term “IL-7 protein” may be used as a concept to include “IL-7 protein and a fragment thereof, wherein the fragment is capable of binding to IL-7 receptor.” IL-7 may be one obtained from humans, rats, mice, monkeys, cows, or sheep.

Unless otherwise specified, the terms “protein”, “polypeptide”, and “peptide” may be used as an interchangeable concept.

Specifically, human IL-7 may have the amino acid sequence represented by SEQ ID NO: 1 (Genbank Accession No. P13232); rat IL-7 may have the amino acid sequence represented by SEQ ID NO: 2 (Genbank Accession No. P56478); mouse IL-7 may have the amino acid sequence represented by SEQ ID NO: 3 (Genbank Accession No. P10168); monkey IL-7 may have the amino acid sequence represented by SEQ ID NO: 4 (Genbank Accession No. NP_001279008); cow IL-7 may have the amino acid sequence represented by SEQ ID NO: 5 (Genbank Accession No. P26895), and sheep TL-7 may have the amino acid sequence represented by SEQ ID NO: 6 (Genbank Accession No. Q28540).

Additionally, the IL-7 protein or a fragment thereof may include variously modified proteins or peptides, i.e., variants. The above modification may be performed by a method of a substitution, a deletion, or an addition of at least one protein to the wild type IL-7, without modifying the function of the TL-7. These various proteins or peptides may have a sequence identify of at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the wild type protein.

Conventionally, a wild type amino acid residue is substituted with alanine, but the substitution may be performed a conservative amino acid substitution, which does not affect or gives a weak effect on the entire protein charge, i.e., polarity or hydrophobicity.

For the conservative amino acid substitution, Table 1 below may be referred to.

TABLE 1

| | |
|---|---|
| Basic | Arginine (Arg, R) |
| | Lysine (Lys, K) |
| | Histidine (His, H) |
| Acidic | Glutamic acid (Glu, E) |
| | Aspartic acid (Asp, D) |
| Uncharged polar | Glutamine (Gln, O) |
| | Asparagine (Asn, N) |
| | Serine (Ser, S) |
| | Threonine (Thr, T) |
| | Tyrosine (Tyr, Y) |
| Non-polar | Phenylalanine (Phe, F) |
| | Tryptophan (Trp, W) |
| | Cystein (Cys, C) |
| | Glycine (Gly, G) |
| | Alanine (Ala, A) |
| | Valine (Val, V) |
| | Proline (Pro, P) |
| | Methionine (Met, M) |
| | Leucine (Leu, L) |
| | Norleucine |
| | Isoleucine |

For each amino acid, additional conservative substitution includes “a homolog” of the amino acid. In particular, the “homolog” refers to an amino acid, in which a methylene group (CH2) is inserted to the side chain of the beta position of the side chain of the amino acid. Examples of the “homolog” may include homophenylalanine, homoarginine, homoserine, etc., but is not limited thereto.

In the structure of the modified IL-7 of A-IL-7, the moiety A may be directly linked to the N-terminal of IL-7, or linked through a linker, and unless otherwise specified, the term may be used as a concept which can be interchangeable with the second domain of IL-7 fusion proteins.

In an embodiment, A may be linked to the N-terminal of IL-7. The A is characterized in that it includes 1 to 10 amino acids, and the amino acid may be selected from the group consisting of methionine, glycine, and a combination thereof. In an embodiment, when A is a single amino acid residue, it is glycine.

Methionine and glycine do not induce immune responses in the human body. The protein therapeutics produced from E. coli always includes methionine in the N-terminal but no adverse reactions have been reported. Also, glycine is widely used as a GS linker and reported that it does not induce immune responses in commercial products as in Dulaglutide (Cell Biophys. 1993 January-June; 22(103):189-224).

In an exemplary embodiment, the A may be an oligopeptide including 1 to 10 amino acids selected from the group consisting of methionine (Met, M), glycine (Gly, G), and a combination thereof. In an embodiment A may be an oligopeptide consisting of 1 to 5 amino acids. For example, the A may have an N-terminal sequence of any one selected from the group consisting of methionine (M), glycine (G), methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, glycine-glycine-glycine, MMMM, MGMM, MGGM, MGGG, MGMG, GMMM, GMGG, GGGG, MMMMM, MMGMM, MMGGM, MGMMG, MMMMG, GGGGG, GGMMM, GGGMG, MGMGMG, MMMGGG, MMGGMM, GGMMGG, MGMGMGMG, MMMMGGGG, MMGGMMGG, MMMMGGGG, MGMGMGMGMG, or and MMMMMGGGGGG. In am embodiment, the A may be methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, or glycine-glycine-glycine.

### Fusion Protein of Modified IL-7 or Modified IL-7 Fusion Protein

Another aspect provides an IL-7 fusion protein, comprising: a first domain comprising a polypeptide having the activity of IL-7 or a similar activity thereof, a second domain including an amino acid sequence having 1 to 10 amino acid residues consisting of methionine, glycine, or a combination thereof, and a third domain which prolongs the half-life of the IL-7 fusion protein.

The third domain may be linked to the N-terminal or the C-terminal of the first domain or the second domain. Additionally, the modified IL-7 including the first domain and the second domain may be linked to both terminals of the third domain.

The third domain may be a fusion partner for increasing in vivo half-life, and preferably, may include any one selected from the group consisting of an Fc region of immunoglobulin or a part thereof, albumin, an albumin-binding polypeptide, Pro/Ala/Ser (PAS), C-terminal peptide (CTP) of β subunit of human chorionic gonadotropin, polyethylene glycol (PEG), long unstructured hydrophilic sequences of amino acids (XTEN), hydroxyethyl starch (HES), an albumin-binding small molecule, and a combination thereof.

When the third domain is an Fc region of immunoglobulin it may be an Fc region of a modified immunoglobulin. In particular, the Fc region of the modified immunoglobulin may be one in which the antibody-dependent cellular cytotoxicity (ADCC) or complement-dependent cytotoxicity (CDC) weakened due to the modification in the binding affinity with the Fc receptor and/or a complement. The modified immunoglobulin may be selected from the group consisting of IgG1, IgG2, IgG3, IgG4, IgA1, IgA2, IgD, IgE and a combination thereof. Specifically, the Fc region of the modified immunoglobulin may include a hinge region, a CH2 domain, and a CH3 domain from the N-terminal to the C-terminal. In particular, the hinge region may include the human IgD hinge region; the CH2 domain may include a part of the amino acid residues of the human IgD and a part of the amino acid residues of the human IgG4 CH2 domain; and the CH3 domain may include a part of the amino acid residues of the human IgG4 CH3 domain.

Additionally, two fusion proteins may form a dimer, for example, when the third domain is an Fc region, the Fc regions may bind to each other and thereby form a dimer.

As used herein, the terms “Fc region”, “Fc fragment”, or “Fc” refers to a protein which includes the heavy chain constant region 2 (CH2) and the heavy chain constant region 3 (CH3) of immunoglobulin but does not include its variable regions of the heavy chain and the light chain and the light chain constant region (CL1), and it may further include a hinge region of the heavy chain constant region. In an embodiment, a hybrid Fc or a hybrid Fc fragment thereof may be called “hFc” or “hyFc.”

Additionally, as used herein, the term “an Fc region variant” refers to one which was prepared by substituting a part of the amino acids among the Fc region or by combining the Fc regions of different kinds. The Fc region variant can prevent from being cut off at the hinge region. Specifically, the 144th amino acid and/or 145th amino acid of SEQ ID NO: 9 may be modified. Preferably, the variant may be one, in which the 144th amino acid, K, was substituted with G or S, and one, in which the 145th amino acid, E, was substituted with G or S.

Additionally, the Fc region or the Fc region variant of the modified immunoglobulin may be represented by the following Formula (I):

N′—(Z1)p-Y—Z2-Z3-Z4-C′. Formula (I)

In the above Formula (I),

N′ is the N-terminal of a polypeptide and C′ is the C-terminal of a polypeptide;

p is an integer of 0 or 1;

Z1 is an amino acid sequence having 5 to 9 consecutive amino acid residues from the amino acid residue at position 98 toward the N-terminal, among the amino acid residues at positions from 90 to 98 of SEQ ID NO: 7;

Y is an amino acid sequence having 5 to 64 consecutive amino acid residues from the amino acid residue at position 162 toward the N-terminal, among the amino acid residues at positions from 99 to 162 of SEQ ID NO: 7;

Z2 is an amino acid sequence having 4 to 37 consecutive amino acid residues from the amino acid residue at position 163 toward the C-terminal, among the amino acid residues at positions from 163 to 199 of SEQ ID NO: 7;

Z3 is an amino acid sequence having 71 to 106 consecutive amino acid residues from the amino acid residue at position 220 toward the N-terminal, among the amino acid residues at positions from 115 to 220 of SEQ ID NO: 8; and

Z4 is an amino acid sequence having 80 to 107 consecutive amino acid residues from the amino acid residue at position 221 toward the C-terminal, among the amino acid residues at positions from 221 to 327 of SEQ ID NO: 8.

Additionally, the Fc fragment may be in the form of having native sugar chains, increased sugar chains, or decreased sugar chains compared to the native form, or may be in a deglycosylated form. The immunoglobulin Fc sugar chains may be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of an embodiment as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

Additionally, the Fc region of the modified immunoglobulin may include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). Additionally, the Fc region of the modified immunoglobulin may include the amino acid sequence of SEQ ID NO: 14 (a non-lytic mouse Fc).

The Fc region of the modified immunoglobulin may be one described in U.S. Pat. No. 7,867,491, and the production of the Fc region of the modified immunoglobulin may be performed referring to the disclosure in U.S. Pat. No. 7,867,491, the entire content of which is incorporated herein by reference.

The second domain may be directly linked to the N-terminal of the first domain or linked by a linker. Specifically, the result may be in the form of the second domain—the first domain or the second domain-linker—the first domain.

The third domain may be directly linked to the first domain or the second domain or linked by a linker. Specifically, the result may be in the form of the second domain—the first domain—the third domain, the third domain—the second domain—the first domain, the second domain—the first domain-linker—the third domain, the third domain-linker—the second domain—the first domain, the second domain-linker—the first domain-linker—the third domain, or the third domain-linker—the second domain—the first domain.

When the linker is a peptide linker, the connection may occur in any linking region. They may be coupled using a crosslinking agent known in the art. Examples of the crosslinking agent may include N-hydroxysuccinimide esters such as 1,1-bis(diazoacetyl)-2-phenylethane, glutaraldehyde, and 4-azidosalicylic acid; imidoesters including disuccinimidyl esters such as 3,3′-dithiobis(succinimidylpropionate), and bifunctional maleimides such as bis-N-maleimido-1,8-octane, but is not limited thereto.

Additionally, the linker may be an albumin linker or a peptide linker. The peptide linker may be a peptide of 10 to 20 amino acid residues consisting of Gly and Ser residues.

When the linker is formed by one selected from the group consisting of a chemical bond, the chemical bond may be a disulfide bond, a diamine bond, a sulfide-amine bond, a carboxy-amine bond, an ester bond, and a covalent bond.

The modified IL-7 may have a structure of A-IL-7 including a polypeptide having the activity of IL-7 or a similar activity thereof and an oligopeptide consisting of 1 to 10 amino acids.

In an embodiment, the modified IL-7 may have an amino acid sequence selected from the group consisting of SEQ ID NOS: 15 to 20. Additionally, the modified TL-7 may have a sequence having a sequence identity of at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the amino acid sequence of SEQ ID NOS: 15, 16, 17, 18, 19, or 20.

In another exemplary embodiment, the modified IL-7 or an IL-7 fusion protein, which comprising a first domain including a polypeptide having the activity of IL-7 or a similar activity thereof, a second domain comprising an amino acid sequence having 1 to 10 amino acid residues consisting of methionine, glycine, or a combination thereof, and a third domain, which is an Fc region of modified immunoglobulin, coupled to the C-terminal of the first domain.

The IL-7 fusion protein may have an amino acid sequence selected from the group consisting of SEQ ID NOS: 21 to 25. Additionally, the IL-7 fusion protein may have a sequence having a sequence identity of at least about 70%, at least about 75%, at least about 80%, 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the amino acid sequence of SEQ ID NOS: 21, 22, 23, 24, or 25.

The IL-7 fusion protein of SEQ ID NOS: 21 to 25 may be encoded by a polynucleotide sequence selected from the group consisting of SEQ ID NOS: 29 to 39.

The nucleic acid molecule may further include a signal sequence or a leader sequence.

As used herein, the term “signal sequence” refers to a fragment directing the secretion of a biologically active molecule drug and a fusion protein, and it is cut off after being translated in a host cell. The signal sequence of an embodiment is a polynucleotide encoding an amino acid sequence initiating the movement of the protein penetrating the endoplasmic reticulum (ER) membrane. The useful signal sequences in an embodiment include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980. 286: 676-683), and other signal sequences know in the art (e.g., see Watson et al., Nucleic Acid Research, 1984. 12:5145-5164).

The characteristics of the signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region.

The central hydrophobic region includes 4 to 12 hydrophobic residues, which immobilize the signal sequence through a membrane lipid bilayer during the translocation of an immature polypeptide. After the initiation, the signal sequence is frequently cut off within the lumen of ER by a cellular enzyme known as a signal peptidase. In particular, the signal sequence may be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., may be used. Additionally, as the secretory signal sequence, the signal sequence included in the wild type IL-7 may be used or it may be used after substituting with a codon with high expression frequency in a host cell.

An isolated nucleic acid molecule encoding the modified IL-7 or an IL-7 fusion protein may be contained in an expression vector.

As used herein, the term “vector” is understood as a nucleic acid means which includes a nucleotide sequence that can be introduced into a host cell to be recombined and inserted into the genome of the host cell, or spontaneously replicated as an episome. The vector may include linear nucleic acids, plasmids, phagemids, cosmids, RNA vectors, virus vectors, and analogs thereof. Examples of the virus vectors may include retroviruses, adenoviruses, and adeno-associated viruses, but are not limited thereto.

As used herein, the term “gene expression” or “expression” of a target protein is understood to refer to transcription of a DNA sequence, translation of an mRNA transcript, and secretion of a fusion protein product or a fragment thereof.

As used herein, the term “host cell” refers to a prokaryotic cell and a eukaryotic cell to which a recombinant expression vector can be introduced. As used herein, the terms “transduced”, “transformed”, and “transfected” refer to the introduction of a nucleic acid (e.g., a vector) into a cell using a technology known in the art.

As used herein, the term “gene expression” or “expression” of a target protein is understood to refer to transcription of a DNA sequence, translation of an mRNA transcript, and secretion of an Fc fusion protein product or an antibody or an antibody fragment thereof.

The useful expression vector may be RcCMV (Invitrogen, Carlsbad) or a variant thereof. The expression vector may include a human cytomegalovirus (CMV) for promoting continuous transcription of a target gene in a mammalian cell and a polyadenylation signal sequence of bovine growth hormone for increasing the stability state of RNA after transcription. In an exemplary embodiment, the expression vector is pAD15, which is a modified form of RcCMV.

The expression vector may be included in an appropriate host cell suitable for the expression and/or secretion of a target protein, by the transduction or transfection of the DNA sequence of an embodiment.

Examples of the appropriate host cell may include, but are not limited to, immortal hybridoma cell, NS/0 myeloma cell, 293 cell, Chinese hamster ovary (CHO) cell, HeLa cell, human amniotic fluid-derived cell (CapT cell), or COS cell.

The modified IL-7 protein and its fusion protein may be produced by a method described in copending application (U.S. application Ser. No. 15/126,313), of which the entire content is incorporated herein by reference.

### Composition

U.S. application Ser. No. 15/773,273, of which entire content is incorporated herein by reference, discloses a formulation containing a modified IL-7 fusion protein. In some embodiments, a pharmaceutical formulation may comprise (a) a modified IL-7 fusion protein; (b) a basal buffer with a concentration of 10 to 50 mM; (c) a sugar with a concentration of 2.5 to 5 w/v %; and (d) a surfactant with a concentration of 0.05 to 6 w/v %. The formulation may further comprise an amino acid, sugar alcohol (e.g., sorbitol, xylitol, maltitol, mannitol, or a mixture thereof). The formulation may have a pH of about 5.

A pharmaceutical composition containing the modified IL-7 or an IL-7 fusion protein may be administered to a subject by a direct administration (e.g., locally by an administration via injection, transplantation, or local administration into a tissue region) or system (e.g., parenterally or orally) via an appropriate means. When the composition is administered parenterally by intravenously, subcutaneously, intraocularly, intraperitoneally, intramuscularly, orally, intrarectally, intraorbitally, intracerebrally, intracranially, intraspinally, intraventricularly, intrathecally, intracistemally, intracapsularly, intranasally, or aerosol administration, the composition preferably contains an aqueous or physiologically applicable suspension of body fluids or a part of the solution thereof. As such, the physiologically acceptable carrier or transporter can be added into the composition and delivered to patients, and this does not cause a negative effect on the electrolyte and/or volume balance of patients. Accordingly, the physiologically acceptable carrier or transporter may be a physiological saline.

Additionally, a DNA construct (or a genomic construct) including a nucleic acid including the modified IL-7 or an IL-7 fusion protein may be used as a part of the gene therapy protocol.

For reconstituting or complementing the functions of a desired protein, an expression vector capable of expressing a fusion protein in a particular cell may be administered along with any biologically effective carrier. This may be any formulation or composition that can efficiently deliver a gene encoding a desired protein or an IL-7 fusion protein into a cell in vivo.

For the purpose of gene therapy using a nucleic acid encoding the modified IL-7 or an IL-7 fusion protein, a subject gene may be inserted into a virus vector, a recombinant bacteria plasmid, or a recombinant eukaryotic plasmid. The virus vector may include a recombinant retrovirus, an adenovirus, an adeno-associated virus, and herpes simplex virus-1, etc., transplanted T-cells, or proliferating the in vitro isolated T-cell aggregates.

### Use of Modified IL-7 or Fusion Protein Thereof and Treatment Regimen

Use of the modified IL-7 or its fusion protein in the preparation of a medicament for use in the treatment of a proliferative disease wherein the medicament comprising a therapeutically effective dose ranging from greater than about 600 μg/kg to about 2,000 μg/kg to be administered twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks to a subject in need thereof.

In an embodiment, use of the modified IL-7 or its fusion protein in the preparation of a medicament for use in the treatment of a proliferative disease wherein the medicament comprising a therapeutically effective dose ranging from greater than about 600 μg/kg to about 2,000 μg/kg to be administered twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks to a subject in need thereof.

Use of the modified IL-7 or its fusion protein for use in the treatment of a proliferative disease comprising administering a compound of formula (I) or a pharmaceutically acceptable salt thereof in a therapeutically effective dosage of about 720 μg/kg or above, about 960 μg/kg or above, about 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein for use in the preparation of a medicament for use in the treatment of a proliferative disease comprising administering a compound of formula (I) or a pharmaceutically acceptable salt thereof in a therapeutically effective dosage of about 720 μg/kg or above, about 960 μg/kg or above, about 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein for use in the treatment of a proliferative disease comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein for use in the preparation of a medicament for use in the treatment of a proliferative disease comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein in the preparation of a medicament for use in increasing a lymphocyte count or lymphocyte production in a subject in need thereof wherein the medicament comprising a therapeutically effective dose ranging from greater than about 600 μg/kg to about 2,000 μg/kg to be administered twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks to the subject.

In an embodiment, use of the modified IL-7 or its fusion protein in the preparation of a medicament for use in increasing a lymphocyte count or lymphocyte production in a subject in need thereof wherein the medicament comprising a therapeutically effective dose ranging from greater than about 600 μg/kg to about 2,000 μg/kg to be administered twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks to the subject.

Use of the modified IL-7 or its fusion protein for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 960 μg/kg or above, about 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

Use of the modified IL-7 or its fusion protein for use in the preparation of a medicament for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 960 μg/kg or above, 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

Use of the modified IL-7 or its fusion protein for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

Use of the modified IL-7 or its fusion protein for use in the preparation of a medicament for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

In an embodiment, the proliferative disease for the above uses is cancer or tumor, which may be, but not limited to, a solid tumor, a cancer of lymphatic system, or leukemia.

The solid tumor may be synovial sarcoma, infiltrating duct carcinoma, rectal cancer, colon cancer, ovary cancer, ascending colon cancer, anal cancer, invasive ductal carcinoma, adenocarcinoma, rectal cancer with paraaortic in metastatis, neuroendocrine carcinoma (cervix), sigmoid colon cancer, or glioblastoma.

In an embodiment, the subject could have previously received or concurrently receives one or more of cancer treatments including surgery, radiation, and chemotherapy.

The (i) modified interleukin-7 or the (ii) interleukin-7 fusion protein, or a pharmaceutical composition containing them may be administered parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

In some aspects, the subject may have a lymphocyte count of 1000 lymphocyte cells or less/μl of blood as determined according to Common Terminology Criteria for Adverse Events (CTCAE) version 4.0. The lymphocyte may be T-cell. The T-cell may include CD4⁺ and/or CD8+ T-cell. For the measurement of lymphocytes in blood sample, whole blood or serum may be used. Therefore, the term “blood” used in relation with the lymphocyte count includes whole blood and/or serum.

In another aspect, the subject has a lymphocyte count of 800 lymphocyte cells or less/μl of blood, a lymphocyte count of about 500 lymphocyte cells or less/μl of blood, or a lymphocyte count of about 200 lymphocyte cells or less/μl of blood.

In an aspect, a number of tumor infiltrating lymphocytes (TILs) in the tumor is increased after the administration compared to a number of TILs in a tumor after administration of the (i) modified interleukin-7 or the (ii) interleukin-7 fusion protein. The TILs may be CD4+ TILs and/or CD8⁺ TILs.

The number of TILs may be increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

In an embodiment, the (i) modified interleukin-7 or the (ii) interleukin-7 fusion protein may be administered twice or more times in an amount of about 720 μg/kg at an interval of about 1 week, about 2 weeks, about 3 weeks, about 4 weeks, about 5 weeks, about 6 weeks, about 7 weeks, or about 8 weeks.

In an embodiment, the (i) modified interleukin-7 or the (ii) interleukin-7 fusion protein may be administered twice or more times in an amount of about 840 μg/kg at an interval of about 2 weeks, about 3 weeks, about 4 weeks, or about 5 weeks, about 6 weeks, about 7 weeks, or about 8 weeks.

In another embodiment, the (i) modified interleukin-7 or the (ii) interleukin-7 fusion protein may be administered twice or more times in an amount of about 960 μg/kg at an interval of about 2 weeks, about 3 weeks, about 4 weeks, about 5 weeks, about 6 weeks, about 7 weeks, about 8 weeks, about 9 weeks, or about 10 weeks.

In still other embodiment, the (i) modified interleukin-7 or the (ii) interleukin-7 fusion protein may be administered twice or more times in an amount of about 1200 μg/kg at an interval of about 3 weeks, about 4 weeks, about 5 weeks, about 6 weeks, about 7 weeks, about 8 weeks, about 9 weeks, about 10 weeks, about 11 weeks, or about 12 weeks.

In still another embodiment, the (i) modified interleukin-7 or the (ii) interleukin-7 fusion protein may be administered twice or more times in an amount of about 1440 μg/kg at an interval of about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 1 month, about 2 months, or about 3 months.

According to an embodiment, the (i) modified interleukin-7 or the (ii) interleukin-7 fusion protein may be administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg, at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, once in 12 weeks, once in 13 weeks, once in 14 weeks, or once in 15 weeks.

According to some aspect, the (i) modified interleukin-7 or the (ii) interleukin-7 fusion protein may be administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, once in 12 weeks, once in 13 weeks, once in 14 weeks, or once in 15 weeks.

According to another aspect, the (i) modified interleukin-7 or the (ii) interleukin-7 fusion protein may be administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, once in 12 weeks, once in 13 weeks, once in 14 weeks, or once in 15 weeks.

According to still another embodiment, the (i) modified interleukin-7 or the (ii) interleukin-7 fusion protein may be administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg, at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, once in 12 weeks, once in 13 weeks, once in 14 weeks, or once in 15 weeks.

In an embodiment, the (i) modified interleukin-7 or the (ii) interleukin-7 fusion protein is administered at a dose of about 650 μg/kg-680 μg/kg, about 680 μg/kg-700 μg/kg, about 700 μg/kg-720 μg/kg, about 720 μg/kg-740 μg/kg, about 740 μg/kg-750 μg/kg, about 750 μg/kg-760 μg/kg, about 760 μg/kg-780 μg/kg, about 780 μg/kg-800 μg/kg, about 800 μg/kg-820 μg/kg, about 820 μg/kg-840 μg/kg, about 840 μg/kg-850 μg/kg, about 850 μg/kg-860 μg/kg, about 860 μg/kg-880 μg/kg, about 880 μg/kg-900 μg/kg, about 900 μg/kg-920 μg/kg, about 920 μg/kg-940 μg/kg, about 940 μg/kg-950 μg/kg, about 950 μg/kg-960 μg/kg, about 960 μg/kg-980 μg/kg, about 980 μg/kg-1000 μg/kg, about 1,000 μg/kg-1,020 μg/kg, about 1,020 μg/kg-1,040 μg/kg, about 1,040 μg/kg-1,060 μg/kg, about 1,060 μg/kg-1,080 μg/kg, about 1,080 μg/kg-1,100 μg/kg, about 1,100 μg/kg-1,120 μg/kg, about 1,120 μg/kg-1,140 μg/kg, about 1,140 μg/kg-1,160 μg/kg, about 1,160 μg/kg-1,180 μg/kg, about 1,180 μg/kg-1,200 μg/kg, about 1,200 μg/kg-1,220 μg/kg, about 1,220 μg/kg-1,240 μg/kg, about 1,240 μg/kg-1,260 μg/kg, about 1,260 μg/kg-1,280 μg/kg, about 1,280 μg/kg-1,300 μg/kg, about 1,300 μg/kg-1,320 μg/kg, about 1,320 μg/kg-1,340 μg/kg, about 1,340 μg/kg-1,360 μg/kg, about 1,360 μg/kg-1,380 μg/kg, about 1,380 μg/kg-1,400 μg/kg, about 1,400 μg/kg-1,420 μg/kg, about 1,420 μg/kg-1,440 μg/kg, about 1,440 μg/kg-1,460 μg/kg, about 1,480 μg/kg, about 1,480 μg/kg-1,500 μg/kg, about 1,500 μg/kg-1,520 μg/kg, about 1,520 μg/kg-1,540 μg/kg, about 1,540 μg/kg-1,560 μg/kg, about 1,560 μg/kg-1,580 μg/kg, about 1,580 μg/kg-1,600 μg/kg, about 1,600 μg/kg-1,620 μg/kg, about 1,620 μg/kg-1,640 μg/kg, about 1,640 μg/kg-1,660 μg/kg, about 1,660 μg/kg-1,680 μg/kg, about 1,680 μg/kg-1,700 μg/kg, about 1,700 μg/kg-1,720 μg/kg, about 1,720 μg/kg-1,740 μg/kg, about 1,740 μg/kg-1,760 μg/kg, about 1,760 μg/kg-1,780 μg/kg, about 1,780 μg/kg-1,800 μg/kg, about 1,800 μg/kg-1,820 μg/kg, about 1,820 μg/kg-1,840 μg/kg, about 1,840 μg/kg-1,860 μg/kg, about 1,860 μg/kg-1,880 μg/kg, about 1,880 μg/kg-1,900 μg/kg, about 1,900 μg/kg-1,920 μg/kg, about 1,920 μg/kg-1,940 μg/kg, about 1,940 μg/kg-1,960 μg/kg, about 1,960 μg/kg-1,980 μg/kg, or about 1,980 μg/kg-1,200 μg/kg at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, once in 12 weeks, once in 13 weeks, or once every 10 days, once every 20 days, once every 30 days, once every 40 days, once every 50 days, once every 60 days, once every 90 days, or once every 100 days.

In an embodiment, the subject may have been received, be under, or will be received chemotherapy. The chemotherapic agent and the modified IL-7 or its fusion protein may be administered simultaneously or sequentially.

### Reference Example 1. Preparation of a Modified IL-7 Protein in which an Oligopeptide is Coupled to IL-7

A modified IL-7, in which an oligopeptide is coupled to the N-terminal of IL-7 was prepared. For the IL-7, the sequence of human IL-7 (SEQ ID NO: 1) was used and, as an oligopeptide, methionine (M), glycine (G), MM, GG, MG, GM, MMM, MMG, MGM, GMM, MGG, GMG, GGM, GGG, DDD, or MMMM sequence was used.

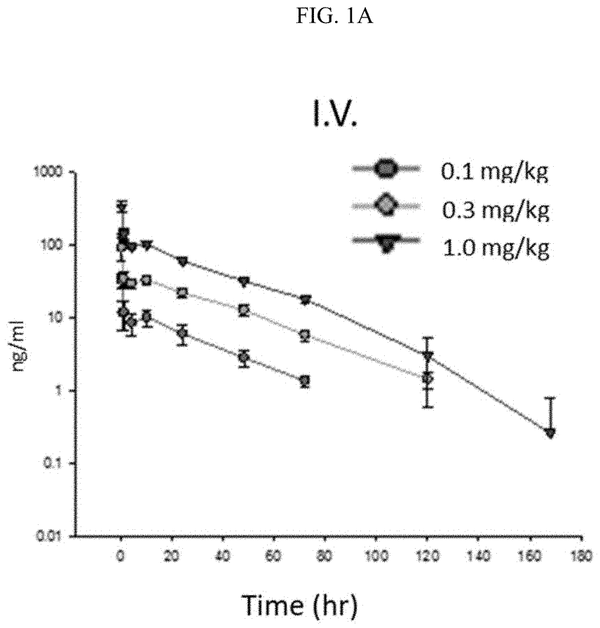

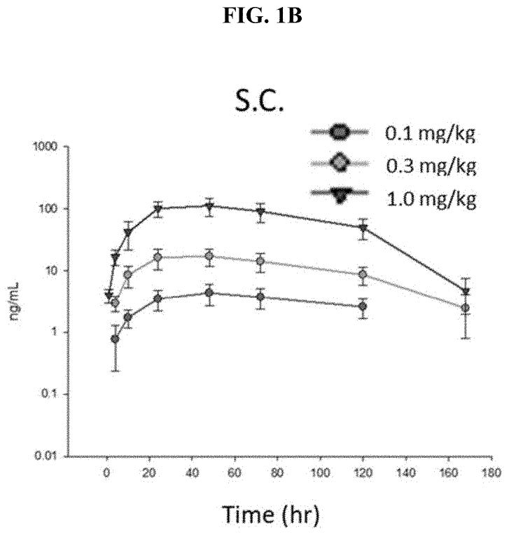

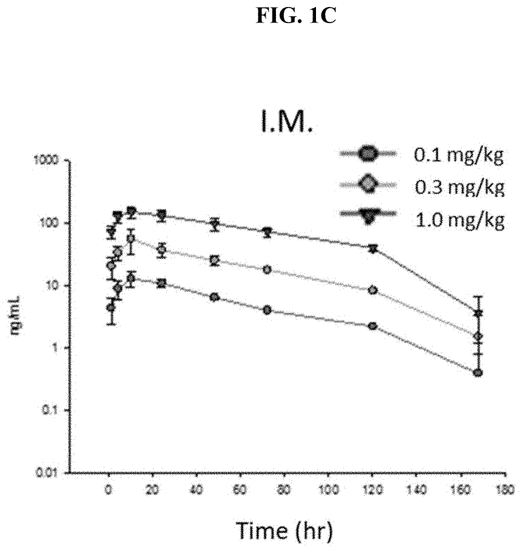

As shown in FIG. 1A, various forms of the modified IL-7 having the structure of the ‘A’-IL-7 were prepared. In this Example, methionine (M), glycine (G), MM, GG, MG, GM, MMM, MMG, MGM, GMM, MGG, GMG, GGM, GGG, DDD, or MMMM sequence was used as the 2nd domain (oligopeptide, ‘A’). Additionally, MGMM, MGGM, MGGG, MGMG, GMMM, GMGG, GGGG, MMMMM, MMGMM, MMGGM, MGMMG, MMMMG, GGGGG, GGMMM, GGGMG, MGMGMG, MMMGGG, MMGGMM, GGMMGG, MGMGMGMG, MMMMGGGG, MMGGMMGG, MMMMGGGG, MGMGMGMGMG, and MMMMMGGGGG are produced as “A.”

For the IL-7 as the 1st domain being fused to the oligopeptide, a nucleic acid sequence of SEQ ID NO: 28 was used. An entire nucleic acid sequence in the form where the IL-7 was fused to the oligopeptide was obtained and then inserted into an expression vector. As a negative control, an IL-7 protein without having the oligopeptide modification was prepared in the same manner.

An expression vector including the A-TL-7 gene was transfected into HEK293 cell. Based on the 300 mL of a suspension culture, a polyplex was prepared using 208.3 ug of DNA and 416.6 ug (μL) of polyethylenimine (PEI)(w/w), and then transfected into the HEK293F cell. Six days after the transfection, the cell culture was obtained and subjected to western blot and thereby the expression rate of the target protein was evaluated. Then, the culture was centrifuged at 8,000 rpm for 30 minutes and the culture debris was removed and filtered using a bottle top filter with a pore size of 0.22 um. As a result, the culture liquid containing the modified IL-7 of M-IL-7, G-IL-7, MM-IL-7, GG-IL-7, MG-IL-7, GM-IL-7, MMM-IL-7, MMG-IL-7, MGM-IL-7, GMM-IL-7, MGG-IL-7, GMG-IL-7, GGM-IL-7, GGG-IL-7, DDD-IL-7, and MMMM-IL-7 was obtained. Additionally, MGMM-IL-7, MGGM-IL-7, MGGG-IL-7, MGMG-IL-7, GMMM-IL-7, GMGG-IL-7, GGGG-IL-7, MMMMM-IL-7, MMGMM-IL-7, MMGGM-IL-7, MGMMG-IL-7, MMMMG-IL-7, GGGGG-IL-7, GGMMM-IL-7, GGGMG-IL-7, MGMGMG-IL-7, MMMGGG-IL-7, MMGGMM-IL-7, GGMMGG-IL-7, MGMGMGMG-IL-7, MMMMGGGG-IL-7, MMGGMMGG-IL-7, MMMMGGGG-IL-7, MGMGMGMGMG-IL-7, and MMMMMGGGGG-IL-7 are produced.

### Reference Example 2. Preparation of an IL-7 Fusion Protein in which an Fc Region is Coupled to the C-Terminal of IL-7

An IL-7 fusion protein, i.e., the second domain—the first domain—the third domain, in which a polypeptide consisting of a heterogeneous amino acid sequence was further coupled to the C-terminal of a modified IL-7 was prepared. For the first domain, the sequence of human IL-7 (SEQ ID NO: 1) was used, and as the second domain, M, G, MM, GG, MG, GM, MMM, MMG, MGM, GMM, MGG, GMG, GGM, GGG, DDD, or MMMM sequence was used. For the third domain, the sequence of the Fc region (SEQ ID NO: 9 or 14) was used. In other embodiments, IL-7—Fc fusion protein wherein MGMM, MGGM, MGGG, MGMG, GMMM, GMGG, GGGG, MMMMM, MMGMM, MMGGM, MGMMG, MMMMG, GGGGG, GGMMM, GGGMG, MGMGMG, MMMGGG, MMGGMM, GGMMGG, MGMGMGMG, MMMMGGGG, MMGGMMGG, MMMMGGGG, MGMGMGMGMG, or MMMMMGGGGG is linked to the N-terminal of the IL-7—Fc are produced.

Various forms of the IL-7 fusion proteins consisting of the second domain, the first domain and the third domain were prepared. In this Example, as the second domain, methionine (M), glycine (G), MM, GG, MG, GM, MMM, MMG, MGM, GMM, MGG, GMG, GGM, GGG, DDD, or MMMM sequence was used; as the first domain, the human IL-7 was used; and as the third domain, hybrid Fc (hFc, hyFc) or mouse non-lytic Fc was used. In additional experiments, MGMM, MGGM, MGGG, MGMG, GMMM, GMGG, GGGG, MMMMM, MMGMM, MMGGM, MGMMG, MMMMG, GGGGG, GGMMM, GGGMG, MGMGMG, MMMGGG, MMGGMM, GGMMGG, MGMGMGMG, MMMMGGGG, MMGGMMGG, MMMMGGGG, MGMGMGMGMG, or MMMMMGGGGG are used as the second domain to produce the modified IL-7 fusion proteins.

In particular, for the hybrid Fc, the hFc (hybrid Fc) disclosed in U.S. Pat. No. 7,867,491, the entire content of which is incorporated by reference herein, was used. The hFc can be coupled to a physiologically active protein and thereby exhibit an excellent in vivo half-life compared to the Fc region of the existing modified immunoglobulin.

A gene expression vector was prepared in the same manner as in Example 1 and transfected, and the cells were cultured to prepare a culture liquid containing various forms of IL-7 fusion proteins. As a result, a culture liquid containing G-IL-7-hyFc, M-IL-7-hyFc, MM-IL-7-hyFc, GG-IL-7-hyFc, MG-IL-7-hyFc, GM-IL-7-hyFc, MMM-IL-7-hyFc, MMG-IL-7-hyFc, MGM-IL-7-hyFc, GMM-IL-7-hyFc, MGG-IL-7-hyFc, GMG-IL-7-hyFc, GGM-IL-7-hyFc, GGG-IL-7-hyFc, DDD-IL-7-hyFc, or MM MM-IL-7-hyFc protein was obtained. MGMM-IL-7-hyFc, MGGM-IL-7-hyFc, MGGG-IL-7-hyFc, MGMG-IL-7-hyFc, GMM M-IL-7-hyFc, GMGG-IL-7-hyFc, GGGG-IL-7-hyFc, MMMMM-IL-7-hyFc, MMGMM-IL-7-hyFc, MMGGM-IL-7-hyFc, MGMMG-IL-7-hyFc, MMMMG-IL-7-hyFc, GGGGG-IL-7-hyFc, GGMMM-IL-7-hyFc, GGGMG-IL-7-hyFc, MGMGMG-IL-7-hyFc, MMMGGG-IL-7-hyFc, MMGGMM-IL-7-hyFc, GGMMGG-IL-7-hyFc, MGMGMGMG-IL-7-hyFc, MMMMGGGG-IL-7-hyFc, MMGGMMGG-IL-7-hyFc, MMMMGGGG-IL-7-hyFc, MGMGMGMGMG-IL-7-hyFc, or MMMMMGGGGG-IL-7-hyFc are produced.

In one aspect, a modified-IL-7—Fc fusion protein with codename GX-I7 (SEQ ID NO: 24) may be used. GX-17 comprises the modified IL-7 of SEQ ID NO: 18 and the hyFc fused to the C-terminal of the modified IL-7.

In an aspect, the IL-7 fusion protein may be administered at a dose of about 60 μg/kg or above. The dose may be in a range from about 60 μg/kg to about 2,000 μg/kg. The dose may be about 60 μg/kg or above, about 120 μg/kg or above, about 240 μg/kg or above, about 480 μg/kg or above, about 720 μg/kg or above, about 960 μg/kg or above, about μg/kg or above, 1,200 μg/kg or above, about 1,700 μg/kg or above, or about 2,000 μg/kg. In another embodiment, the dose may be about 60 μg/kg or above, about 360 μg/kg or above, about 600 μg/kg or above, about 840 μg/kg or above, or about 1440 μg/kg or above.

In an embodiment, the dose may be about 60 μg/kg or above, about 120 μg/kg or above, about 240 μg/kg or above, about 360 μg/kg or above, about 480 μg/kg or above, about 600 μg/kg or above, about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg.

In some aspects, the modified interleukin-7 or the interleukin-7 fusion protein is administered twice or more times in an amount of about 720 μg/kg at an interval of about 1 week, about 2 weeks, about 3 weeks, about 4 weeks, about 5 weeks, about 6 weeks, about 7 weeks, or about 8 weeks. In some aspects, the modified interleukin-7 or the interleukin-7 fusion protein is administered twice or more times in an amount of about 840 μg/kg at an interval of about 2 weeks, about 3 weeks, about 4 weeks, about 5 weeks, about 6 weeks, about 7 weeks, or about 8 weeks. In other aspects, the modified interleukin-7 or the interleukin-7 fusion protein is administered twice or more times in an amount of about 960 μg/kg at an interval of about 2 weeks, about 3 weeks, about 4 weeks, about 5 weeks, about 6 weeks, about 7 weeks, about 8 weeks, or 9 weeks, or about 10 weeks. In some aspects, the modified interleukin-7 or the interleukin-7 fusion protein is administered twice or more times in an amount of about 1200 μg/kg at an interval of about 3 weeks, about 4 weeks, about 5 weeks, about 6 weeks, about 7 weeks, about 8 weeks, about 9 weeks, about 10 weeks, about 11 weeks, or about 12 weeks. In some other aspects, the modified interleukin-7 or the interleukin-7 fusion protein is administered twice or more times in an amount of about 1440 μg/kg at an interval of about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, or about 15 weeks.

### I. Pre-Clinical Study

A. Pharmacokinetic Test after Single Administration of IL-7 Fusion Protein in Rat

In order to observe the pharmacokinetic properties of the IL-7 fusion protein, a pharmacokinetic test was performed using normal rats. Pharmacokinetic tests were conducted by intravenous, subcutaneous and intramuscular administrations. GX-I7 (SEQ ID NO: 24) was used as the IL-7 fusion protein.

Test results for evaluating the in vivo kinetics and bioavailability of the drug when IL-7 fusion protein was administered once intravenously, subcutaneously and intramuscularly to normal rats.

When administered intravenously, AUCₗₐₛₜ and Cₘₐₓ were respectively 349.7 (0.1 mg/kg), 1,440.6 (0.3 mg/kg), 4,225.9 (1.0 mg/kg) h ng/mL and 34.3 (0.1 mg/kg), 86.7 (0.3 mg/kg), 324.5 (1.0 mg/kg) ng/mL, confirming that they increase in a dose-dependent manner. The Tₘₐₓ was 0.083 hours, the terminal T_{1/2} was 22.6, 22.6, and 22.0 hours in each administration group of 0.1, 0.3, and 1.0 mg/kg, and the average terminal T_{1/2} was 22.4 hours. The bioavailability was calculated based on the 72 hr (0.1 mg/kg), 120 hr (0.3 mg/kg), and 168 hr (1.0 mg/kg), which were the time points at which the intravenous administration group's blood concentration can be measured.

When administered subcutaneously, AUCₗₐₛₜ and Cₘₐₓ were 363.8 (0.1 mg/kg), 1,675.9 (0.3 mg/kg), 9,765.4 (1.0 mg/kg) h ng/mL and 4.1 (0.1 mg/kg), 16.4 (0.3 mg/kg), and 105.9 (1.0 mg/kg) ng/mL, respectively. It was confirmed that the Tₘₐₓ was increased to 48.0, 36.0, 48.0 hours in each administration group of 0.1, 0.3, and 1.0 mg/kg, and terminal T_{1/2} was 39.4 and 24.3 hours in each administration group of 0.3 and 1.0 mg/kg. The bioavailability was found to be 104 (0.1 mg/kg), 116 (0.3 mg/kg), and 231 (1.0 mg/kg) based on the blood concentration of the intravenous group.

When administered intramuscularly, AUCₗₐₛₜ and Cₘₐₓ were 732.3 (0.1 mg/kg), 2,898.3 (0.3 mg/kg), 11,027.8 (1.0 mg/kg) h ng/mL and 12.5 (0.1 mg/kg), 51.2 (0.3 mg/kg), and 147.9 (1.0 mg/kg) ng/mL, respectively. It was confirmed that these values increase in a dose-dependent manner. The Tₘₐₓ was about 10.0 hours in all administration groups of 0.1, 0.3, and 1.0 mg/kg, terminal T_{1/2} was 48.3, 39.0, and 25.6 hours in each administration group of 0.1, 0.3, and 1.0 mg/kg. The bioavailability was found to be 209 (0.1 mg/kg), 201 (0.3 mg/kg), and 261 (1.0 mg/kg) based on the blood concentration of the intravenous group.

Interestingly, in this experiment, the modified IL-7 fusion protein administered intramuscularly showed higher bioavailability than intravenous administration, whereas majority of existing drug compounds show lower bioavailability when administered subcutaneously or intramuscularly than intravenous administration. See, FIGS. 1A-1C.

B. Pharmacokinetic Test after Administration of IL-7 Fusion Protein in Monkey

The pharmacokinetic profile in cynomolgus monkey was confirmed through toxicity kinetics after one dose of toxicity test for 4 weeks. After a single administration of 0.6, 2, 6 mg/kg of IL-7 fusion protein, toxicity kinetics were measured for 1 week. As a result, AUCₗₐₛₜ and Cₘₐₓ were 608 (0.6 mg/kg), 2,730 (2 mg/kg), 15,824 (6 mg/kg) h ng/mL and 6 (0.6 mg/kg), 29 (2 mg/kg), 172 (6 mg/kg) ng/mL, showing a dose-dependent increase. Tₘₐₓ was 22-40 hours and terminal T_{1/2} were 164, 90, and 69 hours in each administration group of 0.6, 2, and 6 mg/kg. The terminal T_{1/2} in the 0.6 mg/kg administration group is speculated to be due to the tendency of a slight increase in the blood IL-7 fusion protein concentration in the terminal phase as a biological variation due to a low blood IL-7 fusion protein concentration. Therefore, the average terminal T_{1/2} of the remaining groups except for the 0.6 mg/kg administration group was calculated and was about 80 hours. The results are shown in Table 2.

TABLE 2
Pharmarcokinetic Parameters

| | | | | T_{1/2} | Tₘₐₓ | Cₘₐₓ | AUCₗₐₛₜ | F % |
|---|---|---|---|---|---|---|---|---|
| Animal | | Doses | | (h) | (h) | (ng/mL) | (h · ng/mL) | (0-72 hr) |
| Normal | IV | 0.1 | mg/kg | 22.6 ± 3.1 | 0.083 ± 0.0 | 34.3 ± 4.3 | 349.7 ± 95.5 | 100 |
| Rat | | 0.3 | mg/kg | 22.6 ± 4.3 | 0.083 ± 0.0 | 86.7 ± 33.0 | 1440.6 ± 201.0 | 100 |
| | | 1.0 | mg/kg | 22.0 ± 5.9 | 0.083 ± 0.0 | 324.5 ± 54.5 | 4225.9 ± 193.8 | 100 |
| | SC | 0.1 | mg/kg | ND¹⁾ | 48.0 ± 0.0 | 4.1 ± 1.7 | 363.8 ± 139.9 | 104 |
| | | 0.3 | mg/kg | 39.4 ± 10.7 | 36.0 ± 13.9 | 16.4 ± 5.4 | 1675.9 ± 579.3 | 116 |
| | | 1.0 | mg/kg | 24.3 ± 8.4 | 48.0 ± 12.0 | 105.9 ± 36.5 | 9765.4 ± 3297.6 | 231 |
| | IM | 0.1 | mg/kg | 48.3 ± 12.3 | 10.0 ± 7.0 | 12.5 ± 3.1 | 732.3 ± 112.8 | 209 |
| | | 0.3 | mg/kg | 39.0 ± 6.9 | 10.0 ± 0.0 | 51.2 ± 24.5 | 2898.3 ± 576.3 | 201 |
| | | 1.0 | mg/kg | 25.6 ± 10.3 | 10.0 ± 7.0 | 147.9 ± 18.0 | 11027.8 ± 1591.1 | 261 |
| cynomolgus | SC | 0.6 | mg/kg | 164 ± 106 | 40 ± 63 | 6 ± 2 | 608 ± 156 | — |
| monkey | | 2 | mg/kg | 90 ± 43 | 22 ± 24 | 29 ± 6 | 2730 ± 712 | — |
| | | 6 | mg/kg | 69 ± 37 | 26 ± 23 | 172 ± 50 | 15,824 ± 2700 | — |

¹⁾ND: Not detected

### C. Safety Test Using Normal Rat and Monkey

To set the dose for repeated administration toxicity of the IL-7 fusion protein, the test was conducted for 2 weeks using normal rats and cynomolgus monkeys, and (sub)chronic toxicity was measured according to repeated administrations at the set doses and the duration of drug administration. Repeated toxicity tests were performed for 4 weeks to evaluate the deterioration, delayed occurrence, and reversibility of the toxicity phenomena. The safety pharmacology test was conducted at doses of 0, 1.2, 4, 12 mg/kg of IL-7 fusion protein (GX-I7) in normal rats and 0, 0.6, 2, 6 mg/kg in cynomolgus monkey. In both normal rats and cynomolgus monkeys, all evaluated doses of IL-7 fusion protein (GX-I7) showed no significant effects on general behavior, nervous system, respiratory system, and cardiovascular system of the tested animals.

When IL-7 fusion protein (GX-I7) was administered subcutaneously in normal rats once a week, a total of 5 times for 4 weeks, even at doses up to 12 mg/kg/week, no toxicological findings caused by drugs were observed. Although symptoms related to the test drug were observed, no drug exposure in blood was observed after repeated administrations. This seems to be the shielding effect by the drug-specific antibody (anti-drug antibody), and as a result, the maximum non-toxic dose (NOAEL) for the administration of IL-7 fusion protein (GX-I7) once a week for 4 weeks in normal rats could not be determined. In the 4 week repeated subcutaneous toxicity test in Cynomolgus Monkey, no drug toxicity was observed even at doses up to 6 mg/kg/week, so the maximum non-toxic dose (NOAEL) of IL-7 fusion protein (GX-I7) was set at 6 mg/kg/week. This corresponds to about 2 mg/kg when converted to a human equivalent dose (HED).

### II. Clinical Study

### A. Objective and Demographics

This clinical trial was conducted on (a) patients with locally advanced or metastatic solid cancer and (b) glioblastoma patients with severe-treatment-related lymphopenia after adjuvant chemotherapy. IL-7 fusion protein was administered at intervals of every 3 weeks, every 4 weeks, every 6 weeks, every 8 weeks, every 9 weeks, or every 12 weeks, and safety, tolerability, and absolute lymphocyte count increasing effects were evaluated.

(1) All subjects enrolled in the dose-escalation phase of metastatic solid cancer patient group were previously failed with existing standard treatment or histologically diagnosed with local-progressive, recurrent or metastatic non-curable solid cancer. The patients were verified through the history of cancer diagnosis and related treatment, and it was confirmed that it met the selection/exclusion criteria of this study through other medical history and screening tests. The 21 enrolled subjects were composed of Colon Cancer 10 (47.6%), Rectal Cancer 5 (23.8%), Breast Cancer 2 (9.5%), Ovary Cancer 1 (4.7%), Synovial Sarcoma 1 (4.7%), Anal Cancer 1 (4.7%), and Cervical Cancer 1 (4.7%). Based on all safety, pharmacokinetic/pharmacokinetic/immunogenic analysis results collected, the dose to be evaluated at the dose-expansion stage (recommended Phase 2 dose, RP2D) and administration intervals were selected.

(2) The clinical study for glioblastoma patients was carried out to select a Recommended Phase 2 dose (RP2D), based on safety, pharmacodynamic/immunogenicity analysis results from the patients enrolled in the Cohort 1-5 dose-escalation stage (a total of 15 patients). Subjects without serious toxicity and without clinical significance of disease progression continued to administer the test drug based on the investigator's clinical significance and risk assessment.

The age of the participating patients ranges from 32 to 81 years old and include both female and male. The patients were previously diagnosed for synovial sarcoma, infiltrating duct carcinoma, rectal cancer, colon cancer, ovary cancer, ascending colon cancer, anal cancer, invasive ductal carcinoma, adenocarcinoma, rectal cancer with paraaortic in metastatis, neuroendocrine carcinoma (cervix), sigmoid colon cancer, or glioblastoma. The participating patients have previously received one or more of cancer treatments including surgery, radiation, and chemotherapy.

### B. Study Design

Patients with locally advanced or metastatic solid cancer and glioblastoma enrolled in the Dose Escalation Stage (21, 28, 42, 56, 63, or 84 days per 1 cycle) were administered at intervals of 21, 28, 42, 56, 63, or 84 days with a fixed dose of the modified IL-7 fusion protein (GX-I7) intramuscularly on the first day of each cycle.

Dose escalation stage includes intramuscular administrations of the IL-7 fusion protein in 9 stages (or 5 stages) doses (60, 120, 240, 480, 720, 960, 1,200, 1,700, and 2,000 μg/kg; or 60, 360, 600, 840, 1,440 μg/kg) as shown in Table 3, and safety, tolerability and pharmacokinetics/pharmacodynamic changes were evaluated. During intramuscular injection, the injection site is divided and administered so that the injection volume does not exceed 2 mL per injection site.

TABLE 3
Dosing Group and Dose Escalation

| Subjects with Solid Tumor | | Subjects with glioblastoma | | |
|---|---|---|---|---|
| | Dose | | Dose | Number of |
| Cohort | (μg/kg) | Cohort | (μg/kg) | Subjects |
| Cohort 1 | 60 | Cohort 1 | 60 | 3 + 3 |
| Cohort 2 | 120 | — | — | 3 + 3 |
| Cohort 3 | 240 | — | — | 3 + 3 |
| — | — | Cohort 2 | 360 | 3 + 3 |
| Cohort 4 | 480 | — | — | 3 + 3 |
| — | — | Cohort 3 | 600 | 3 + 3 |
| Cohort 5 | 720 | — | — | 3 + 3 |
| — | — | Cohort 4 | 840 | 3 + 3 |
| Cohort 6 | 960 | — | — | 3 + 3 |
| Cohort 7 | 1,200 | — | — | 3 + 3 |
| — | — | Cohort 5 | 1,440 | 3 + 3 |
| Cohort 8 | 1,700 | — | — | 3 + 3 |
| Cohort 9 | 2,000 | — | — | 3 + 3 |

### C. Safety

1. Patients with Solid Tumor

The safety was evaluated through adverse reactions (abnormal laboratory test values, clinical symptoms and signs that subjects complained of, researcher evaluation, etc.) of dose-escalation stage subjects (a total of 21 patients with locally advanced or metastatic solid cancer). Results are shown in Table 4.

Of the total adverse reactions, 44 cases of adverse drug reactions (ADRs) judged as “relevant to the test drug” occurred in 16 out of 21 (76.2%). When grades were classified according to NCI-CTCAE (version 4.0) for the severity of adverse drug reactions (ADR), 29 cases were mild (Grade 1) and 15 cases were moderate (Grade 2), and no ADR of grade 3, 4, 5 was reported. There were a total of 3 reported Serious Adverse Events (SAEs), all of which were evaluated as “no relationship with the test drug.” According to the reported frequency of ADR, injection site reactions occurred most frequently, with 25 cases reported in 14 out of 21 (66.7%), Pyrexia 8 cases, Rash or Rash popular 4 cases, Decreased appetite 2 cases, and 1 case of each Asthenia, Back pain, Constipation, Influenza like illness, and Myalgia were reported. Accordingly, it was confirmed that there were no serious adverse reactions caused by the drug.

TABLE 4
Total Adverse Events and Adverse Drug Reactions in Solid Tumor Patients

| | | GX-I7 (hIL-7-hyFc) | | | |
|---|---|---|---|---|---|
| n(%), | | 60 μg/kg | 120 μ/kg | 240 μ/kg | 480 μ/kg |
| [case] | | (n = 3) | (n = 3) | (n = 3) | (n = 3) |
| Any TEAE* | | 3(100.0), [13] | 3(100.0), [29] | 3(100.0), [14] | 3(100.0), [18] |
| ADR | | 3(100.0), [6] | 2(66.7), [9] | 3(100.0), [6] | 2(66.7), [6] |
| Severity | Gr1 | 2(66.7), [4] | 2(66.7), [7] | 3(100.0), [4] | 2(66.7), [4] |
| | Gr2 | 1(33.3), [2] | 2(66.7), [2] | 2(66.7), [2] | 2(66.7), [2] |
| | Gr3 | — | — | — | — |
| | Gr4 | — | — | — | — |
| | Gr5 | — | — | — | — |

ADR by Preferred term

| | | | | |
|---|---|---|---|---|
| Injection site | 3(100.0), [6] | 2(66.7), [5] | 3(100.0), [3] | 2(66.7), [4] |
| reaction | | | | |
| Pyrexia | | 1(33.3), [1] | 1(33.3), [1] | 1(33.3), [2] |
| Rash/Rash | | 1(33.3), [1] | — | — |
| papular | | | | |
| Decreased | | 1(33.3), [1] | — | — |
| appetite | | | | |
| Asthenia | — | — | 1(33.3), [1] | — |
| Back pain | | 1(33.3), [1] | — | — |
| Constipation | — | — | — | — |
| Influenza like | — | — | — | — |
| illness | | | | |
| Myalgia | — | — | 1(33.3), [1] | — |

GX-I7 (hIL-7-hyFc)

| n(%), | | 720 μ/kg | 960 μ/kg | 1200 μ/kg | Total |
|---|---|---|---|---|---|
| [case] | | (n = 3) | (n = 3) | (n = 3) | (n = 21) |
| Any TEAE* | | 3(100.0), [18] | 3(100.0), [4] | 3(100.0), [14] | 21(100.0), [110] |
| ADR | | 1(33.3), [5] | 2(66.7), [2] | 3(100.0), [10] | 16(76.2), [44] |
| Severity | Gr1 | 1(33.3), [2] | 1(33.3), [1] | 2(66.7), [7] | 13(61.9), [29] |
| | Gr2 | 1(33.3), [3] | 1(33.3), [1] | 3(100.0), [3] | 12(57.1), [15] |
| | Gr3 | — | — | — | — |
| | Gr4 | — | — | — | — |
| | Gr5 | — | — | — | — |

ADR by Preferred term

| | | | | |
|---|---|---|---|---|
| Injection site | 1(33.3), [3] | — | 3(100.0), [4] | 14(66.7), [25] |
| reaction | | | | |
| Pyrexia | — | 1(33.3), [1] | 2(66.7), [3] | 6(28.6), [8] |
| Rash/Rash | 1(33.3), [1] | 1(33.3), [1] | 1(33.3), [1] | 4(19.0), [4] |
| papular | | | | |
| Decreased | — | — | 1(33.3), [1] | 2(9.5), [2] |
| appetite | | | | |
| Asthenia | — | — | | 1(4.8), [1] |
| Back pain | — | — | | 1(4.8), [1] |
| Constipation | 1(33.3), [1] | — | | 1(4.8), [1] |
| Influenza like | — | — | 1(33.3), [1] | 1(4.8), [1] |
| illness | | | | |
| Myalgia | — | — | | 1(4.8), [1] |

*TEAE: Treatment emergent adverse event

2. Glioblastoma Patients

Safety was evaluated through adverse reactions (abnormal laboratory test values, clinical symptoms and signs that subjects complained of, investigator evaluation, etc.) of 15 glioblastoma patients, reported during the dose-increment stage. See Table 5.

Of the total adverse reactions, 32 cases of adverse drug reactions (ADRs) judged as “relevant to the test drug” occurred in 13 out of 15 subjects (86.7%). In each group, one patient in the 60 μg/kg group (33.3%, 2 cases), 3 patients in the 360 μg/kg group (100.0%, 10 cases), and 3 patients in the 600 μg/kg group. (100.0%, 5 cases), 3 patients (100.0%, 7 cases) in the 840 μg/kg group, and 3 patients (100.0%, 8 cases) in the 1,440 μg/kg group. When grades were classified according to NCI-CTCAE (version 4.0) for the severity of adverse drug reactions (ADR), 21 cases were mild (Grade 1) and 11 cases were moderate (Grade 2), and no ADR of grades 3, 4, 5 was reported. According to the reported frequency of ADR, injection site reactions occurred most frequently, with 9 cases reported in 11 out of 15 cases (66.0%). Urticaria 6 cases, Pruritus 3 cases, and 2 cases for each of Feeling hot, Pyrexia, and Swelling as well as 1 case for each of injection site pain, Myalgia, Oedema peripheral, Fatigue, and Rash were reported. Accordingly, it was confirmed that there were no serious adverse reactions caused by the drug.

TABLE 5
Total Adverse Events and Adverse Drug Reactions in Glioblastoma Patients

| | | GX-I7 (modified IL-7 - Fc fusion protein) | | | | | |
|---|---|---|---|---|---|---|---|
| n(%), | | 60 μ/kg | 360 μ/kg | 600 μ/kg | 840 μ/kg | 1,440 μ/kg | Total |
| [case] | | (n = 3) | (n = 3) | (n = 3) | (n = 3) | (n = 3) | (n = 15) |
| Any TEAE* | | 3(100.0), [22] | 3(100.0), [22] | 3(100.0), [24] | 3(100.0), [18] | 3(100.0), [9] | 15(100.0), [95] |
| ADR | | 1(33.3), [2] | 3(100.0), [10] | 3(100.0), [5] | 3(100.0), [7] | 3(100.0), [8] | 13(86.7), [32] |
| Severity | Gr1 | 1(33.3), [1] | 3(100.0), [7] | 3(100.0), [5] | 2(66.7), [2] | 2(66.7), [6] | 11(73.3), [21] |
| | Gr2 | 1(33.3), [1] | 1(33.3), [3] | 0(0.0), [0] | 3(100.0), [5] | 2(66.7), [2] | 8(53.3), [11] |
| | Gr3 | | | | | | |
| | Gr4 | | | | | | |
| | Gr5 | | | | | | |

ADR by Preferred term

| | | | | | | |
|---|---|---|---|---|---|---|
| Injection site | 1(33.3), [1] | 3(100.0), [5] | 1(33.3), [1] | 2(66.7), [2] | 2(66.7), [2] | 9(66.0), [11] |
| reaction | | | | | | |
| Injection site | 0(0.0), [0] | 0(0.0), [0] | 1(33.3), [1] | 0(0.0), [0] | 0(0.0), [0] | 1(6.7), [1] |
| pain | | | | | | |
| Urticaria | 1(33.3), [1] | 1(33.3), [1] | 2(66.7), [2] | 1(33.3), [1] | 1(33.3), [1] | 6(66.7), [6] |
| Pruritus | 0(0.0), [0] | 0(0.0), [0] | 0(0.0), [0] | 1(33.3), [1] | 2(66.7), [2] | 3(20.0), [3] |
| Feeling hot | 0(0.0), [0] | 1(33.3), [1] | 0(0.0), [0] | 0(0.0), [0] | 1(33.3), [1] | 2(13.3), [2] |
| Pyrexia | 0(0.0), [0] | 1(33.3), [1] | 0(0.0), [0] | 0(0.0), [0] | 1(33.3), [1] | 2(13.3), [2] |
| Swelling | 0(0.0), [0] | 0(0.0), [0] | 0(0.0), [0] | 1(33.3), [1] | 1(33.3), [1] | 2(13.3), [2] |
| Myalgia | 0(0.0), [0] | 1(33.3), [1] | 0(0.0), [0] | 0(0.0), [0] | 0(0.0), [0] | 1(6.7), [1] |
| Oedema | 0(0.0), [0] | 1(33.3), [1] | 0(0.0), [0] | 0(0.0), [0] | 0(0.0), [0] | 1(6.7), [1] |
| peripheral | | | | | | |
| Fatigue | 0(0.0), [0] | 0(0.0), [0] | 1(33.3), [1] | 0(0.0), [0] | 0(0.0), [0] | 1(6.7), [1] |
| Rash | 0(0.0), [0] | 0(0.0), [0] | 0(0.0), [0] | 1(33.3), [2] | 0(0.0), [0] | 1(6.7), [2] |

*TEAE; Treatment emergent adverse event

3. Pharmacokinetics

For pharmacokinetic parameters, values were calculated for each subject, and their mean, standard deviation, minimum, and maximum were presented as descriptive statistics for each dose group.

Blood was collected before (0 hours) and 0.5, 6, 12, 24, 48, 72, 168, 336, and 504 hours after administration of the modified IL-7 fusion protein GX-I7, and the concentration of IL-7 in the blood was analyzed using a commercially available ELISA kit (Human IL-7 Quantikine HS ELISA Kit HS750 from R&D Systems), and the results are shown in tables (PK parameters) and graphs.

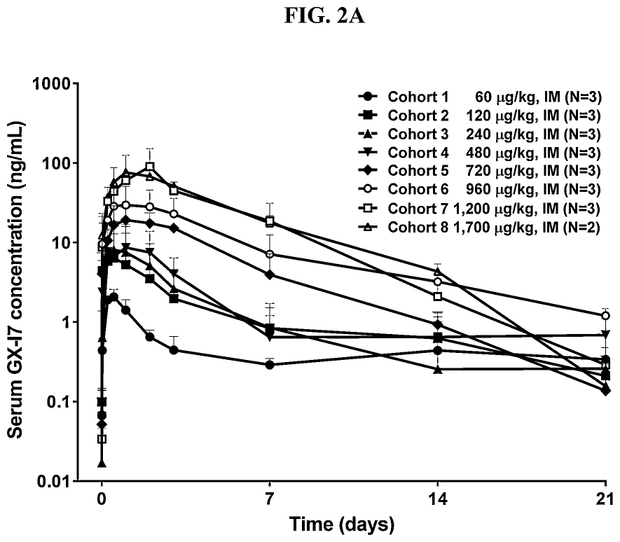

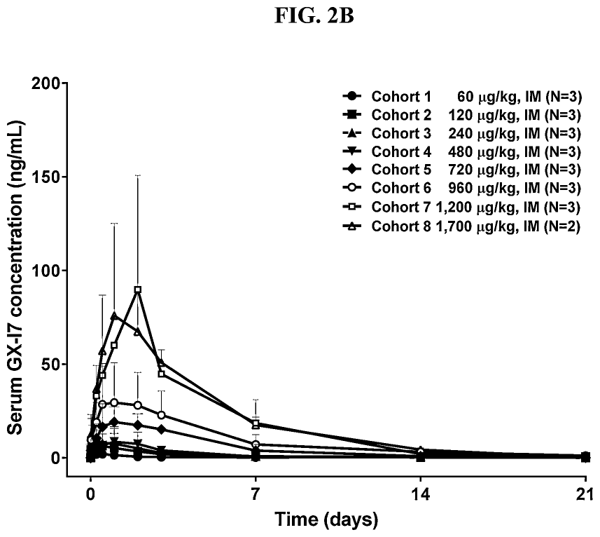

The blood concentration-time curve for each subject in each dose group is shown in [FIG. 2], and the pharmacokinetic parameters are presented in Table 6. The results of blood concentration measurements according to intramuscular administration of IL-7 fusion protein, before and after administration in all dose groups. It was observed that the blood concentration of IL-7 increased in a dose-dependent manner.

At the first intramuscular administration, the blood concentration of the modified IL-7 fusion protein decreased after reaching the highest blood concentration within an average of 12 to 48 hours for each dose, and the half-life (t_{1/2}) was 33 to 147 hours. There was a difference between.

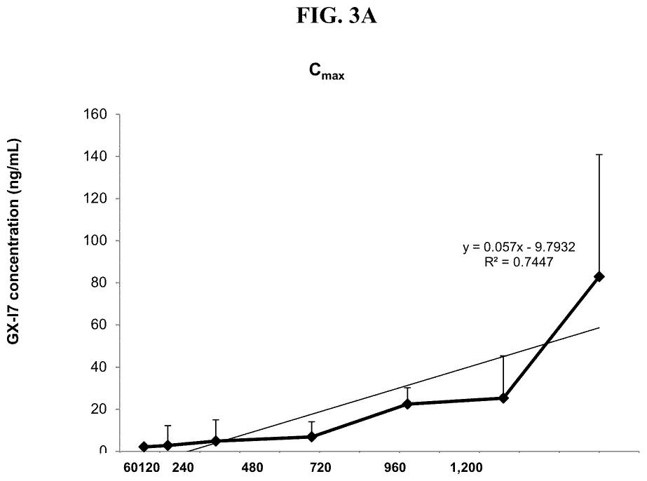

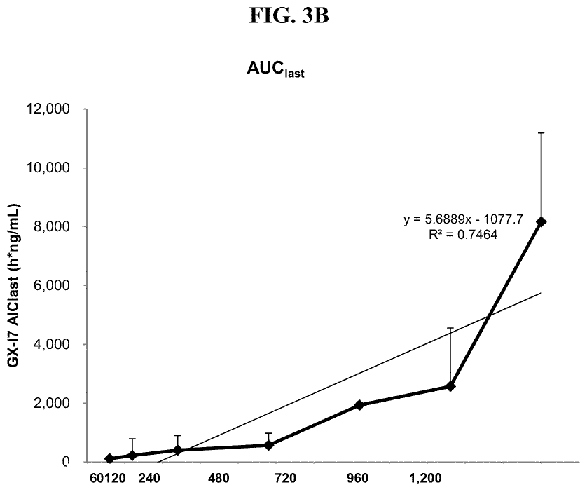

Although there are differences between individuals, Cmax and AUClast tended to increase as the dose of the test drug increased, and Cₘₐₓ and AUCₗₐₛₜ increased more than the dose increased at 1,200 μg/kg than at 960 μg/kg. Showed a pattern. The correlation between Cₘₐₓ and AUCₗₐₛₜ change by dose is presented in FIGS. 3A and 3B.

TABLE 6
Summary of Serum IL-7 fusion protein PK Parameters
after Single Intramuscular Injections

| | Dose | tₘₐₓ¹⁾ | Cₘₐₓ²⁾ | AUC_{0-168 hr}²⁾ | AUCₗₐₛₜ²⁾ |
|---|---|---|---|---|---|
| Cohort | (μg/kg) | (h) | (ng/mL) | (h*ng/mL) | (h*ng/mL) |
| Cohort 1 | 60 | 12 (6, 12) | 2.08 ± 0.47 | 109.1 ± 13.2 | 207.8 ± 131.2 |
| (n = 3) | | | | | |
| Cohort 2 | 120 | 12 (12, 72) | 2.74 ± 9.42 | 221.5 ± 570.4 | 380.0 ± 722.4 |
| (n = 3) | | | | | |
| Cohort 3 | 240 | 48 (12, 48) | 4.89 ± 10.04 | 402.6 ± 499.5 | 523.1 ± 534.8 |
| (n = 3) | | | | | |
| Cohort 4 | 480 | 24 (6, 24) | 6.87 ± 7.17 | 565.1 ± 413.6 | 821.5 ± 314.1 |
| (n = 3) | | | | | |
| Cohort 5 | 720 | 48 (12, 72) | 22.4 ± 7.77 | 1932.9 ± 96.4 | 2309.2 ± 119.7 |
| (n = 3) | | | | | |
| Cohort 6 | 960 | 24 (12, 72) | 25.2 ± 20.1 | 2567.0 ± 1990.7 | 3444.3 ± 2300.3 |
| (n = 3) | | | | | |
| Cohort 7 | 1200 | 24 (24, 48) | 82.9 ± 58.0 | 6858.9 ± 2442.8 | 8161.7 ± 3027.7 |
| (n = 3) | | | | | |
| Cohort 8 | 1700 | 36 (24, 48) | 75.8 ± 41.6 | 7220.6 ± 1773.3 | 7875.1 ± 2923.2 |
| (n = 2) | | | | | |

[Note]
Cₘₐₓ = maximum concentration;
tₘₐₓ = time to maximum concentration;
AUC_{0-168 h} = area under the curve from time zero to the 168 h time point;
¹⁾Median (min, max),
²⁾Geomean ± SD

4. Pharmacodynamic (PD) Characteristics

(a) Clinical Trial for Solid Cancer Patients

In order to evaluate the biomarker that can be applied as an exploratory pharmacodynamic indicator for the activity of the IL-7 fusion protein in patients with locally advanced or metastatic solid cancer, the test drug was administered to 21 subjects enrolled in the dose-escalation stage. Peripheral blood was collected before and after, and various immune cell types and percentage changes in the blood were measured.

(b) Clinical Trial for Patients with Glioblastoma

Peripheral before and after administration of the test drug in 15 subjects enrolled in the dose-escalation stage for the evaluation of biomarkers that can be applied as exploratory pharmacodynamic indicators for the activity of IL-7 fusion protein in glioblastoma patients. Blood was collected, and various immune cell types and ratio changes in the blood were measured.

4.1 Changes in Absolute Lymphocyte Count (ALC) by Administration of IL-7 Fusion Protein

In order to measure the absolute lymphocyte count (ALC), according to each dose group (Cohort) for the entire patient group, the patient's blood was collected before and 3 weeks after the administration of IL-7 fusion protein. The absolute value of ALC was measured using an analyzer.

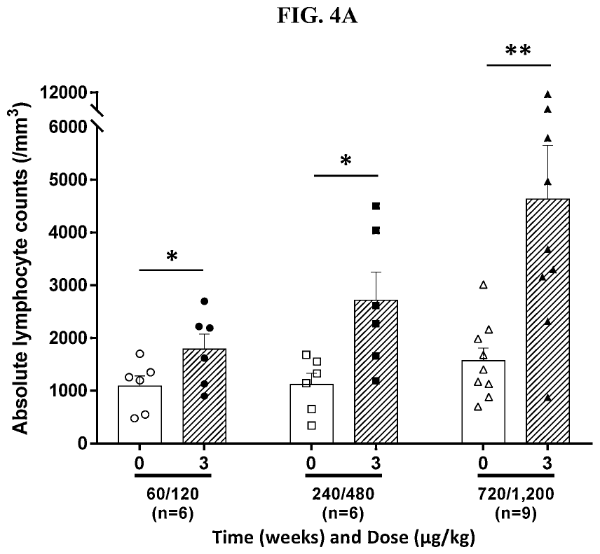

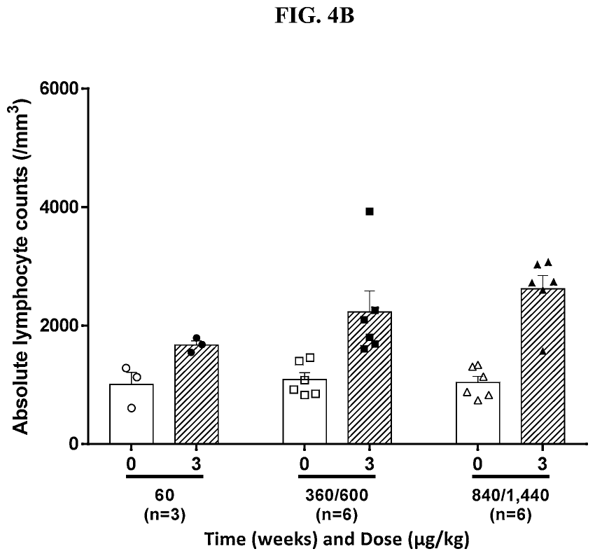

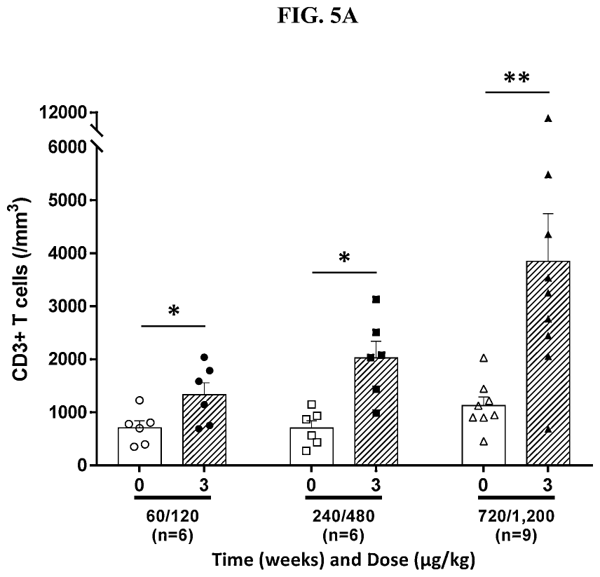

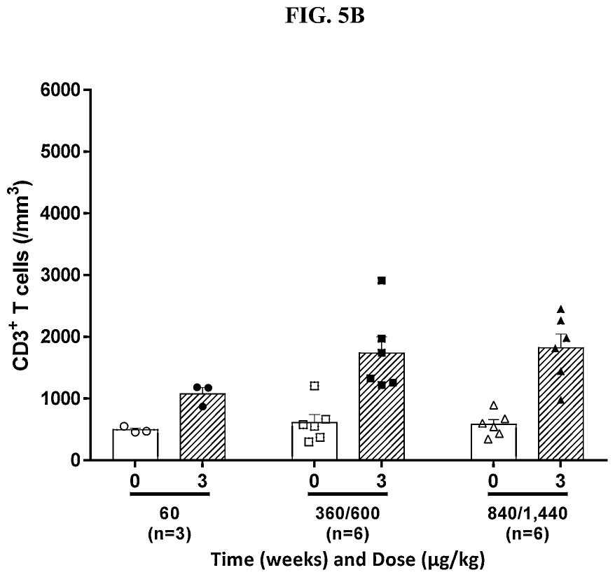

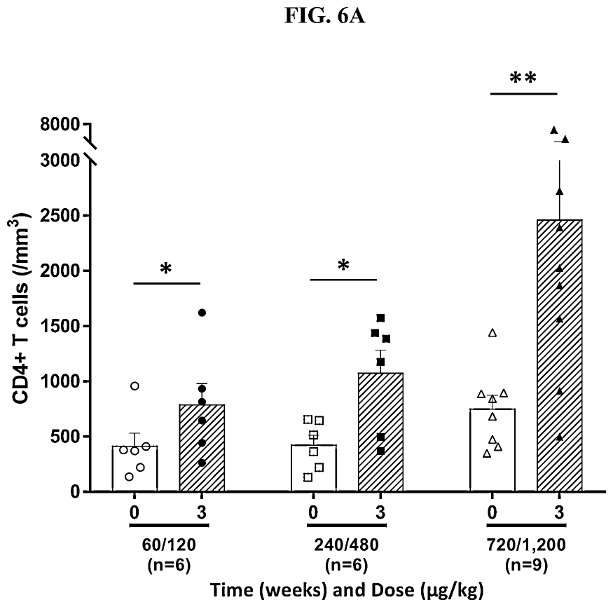

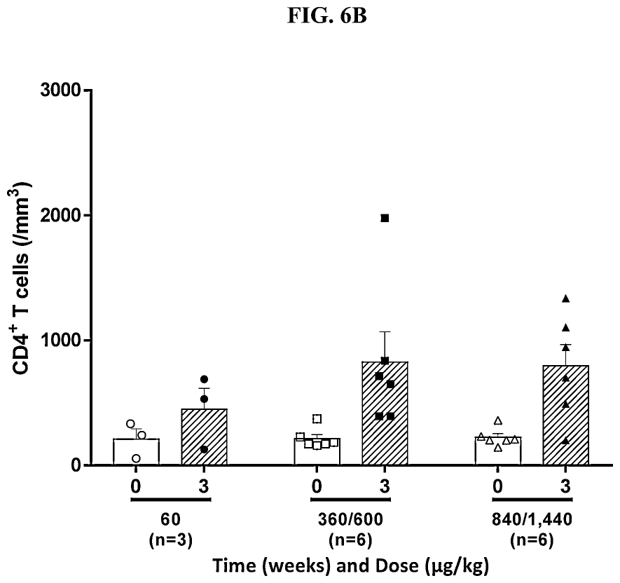

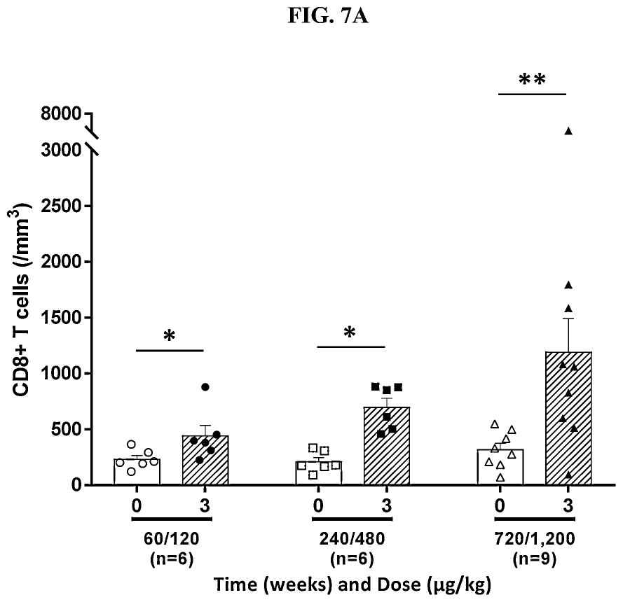

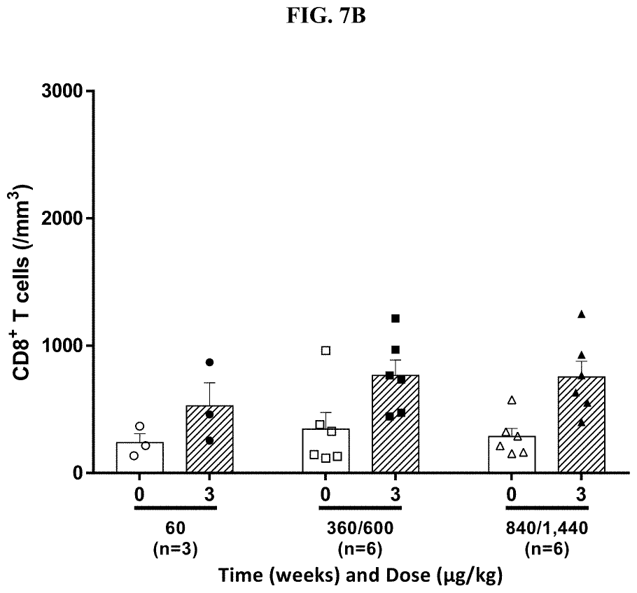

Solid cancer and glioblastoma patient group at low dose (60-120 μg/kg & 60 μg/kg), medium dose (240-480 μg/kg & 360-600 μg/kg), high dose (720-1,200 μg/kg & 840-1,440 μg/kg) groups, and the change in absolute ALC values for 3 weeks from the baseline is presented in FIGS. 4A and 4B. As a result, it was confirmed that in the high dose group (720-1,440 μg/kg), the change in absolute value of ALC increased up to 4.4 times compared to the baseline value. This pattern was confirmed to show the same increase pattern in CD3*, CD4*, and CD8⁺ T cells. See FIGS. 5A, 5B, 6A, 6B, 7A, and 7B.

4.2 Changes in ALC in Patients with Lymphopenia

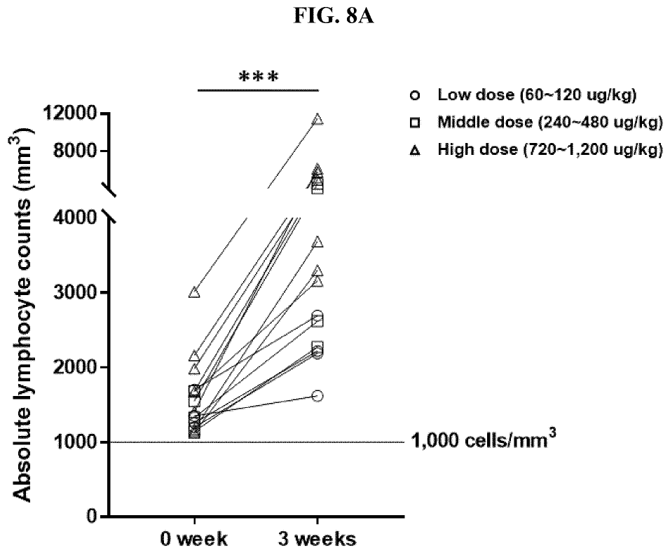

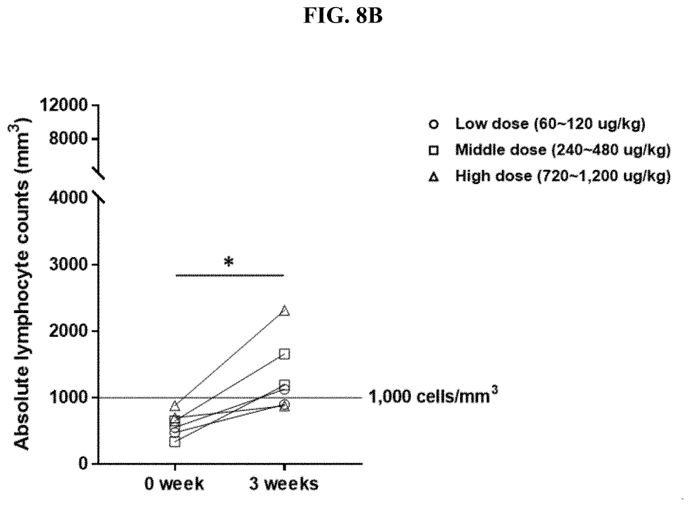

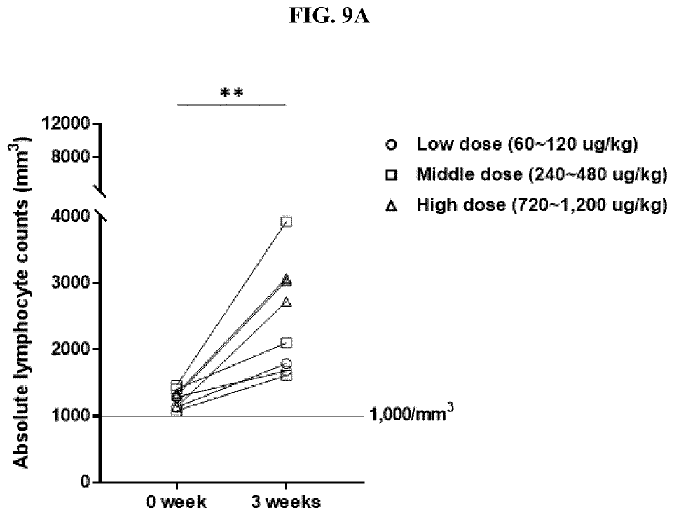

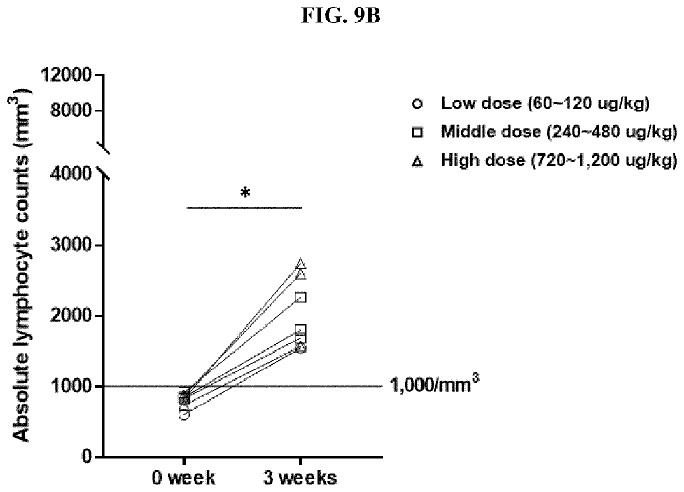

For all solid cancer patients and glioblastoma patients, patients with baseline ALC levels lower than 1,000 cells/mm3 prior to IL-7 fusion protein administration were classified as lymphopenia patients, and IL-7 fusion protein was administered at intervals of every 3 weeks or 6 weeks (solid cancer) and every 4 weeks, every 8 weeks, every 9 weeks, or every 12 weeks (GBM). Afterwards, changes in ALC were observed for each group of patients with lymphopenia and non-lymphopenia and the results are shown in FIGS. 8A, 8B, 9A, and 9B.

As a result of the analysis, it was confirmed that the ALC value increased significantly in the non-lymphopenia patient group, and the ALC value after 3 weeks of administration increased to within the normal range in the lymphopenia patient group with ALC lower than 1,000 cells/mm³. In addition, it was confirmed that the magnitude of the change compared to the baseline value in the non-lymphopenia patient group increased similarly in the lymphopenia patient group, especially in the high dose group.

4.3 Ki67, CD127 (IL-7Ra) and T Cell Subtypes (Subsets) Analysis Results

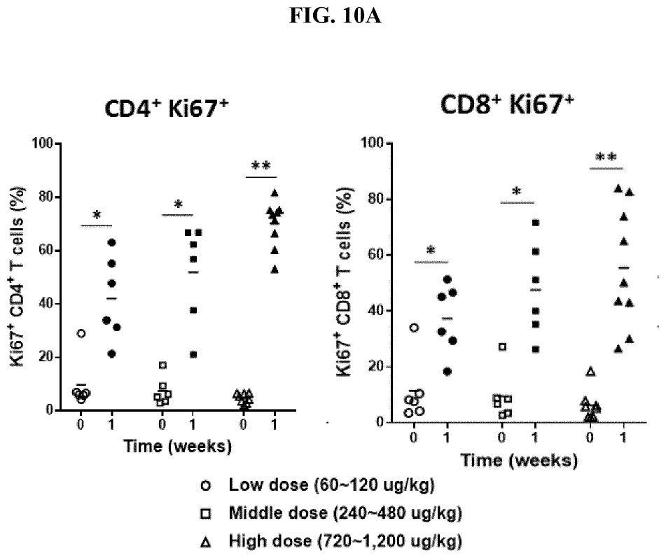

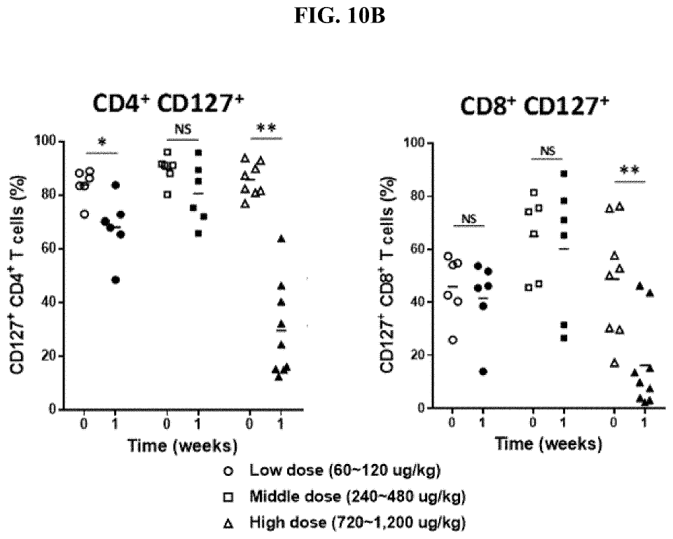

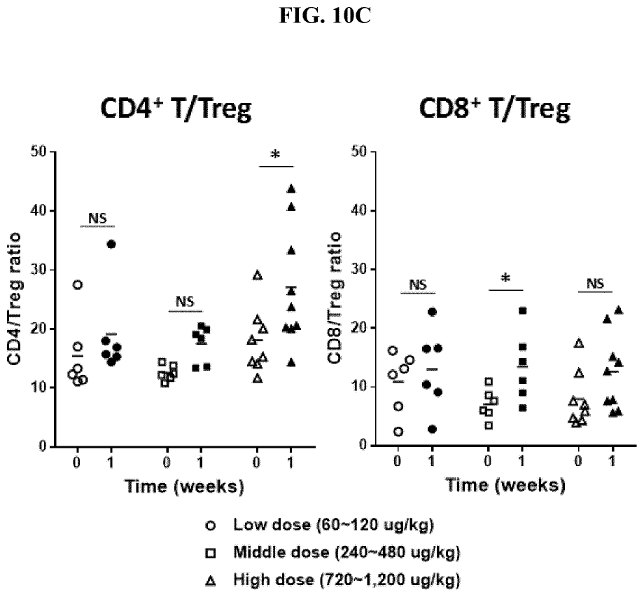

From the changes in Ki67 expression as a leading indicator of the increase in ALC in solid cancer patients, it was confirmed that the amount of Ki67 expression in both CD4⁺ T cells and CD8⁺ T cells was increased in a dose-dependent matter in both of CD4⁺ T cells and CD8⁺ T cells. On the other hand, it was confirmed that the expression level of CD127, the receptor of IL-7, significantly decreased compared to the baseline level at high doses. In addition, the changes in the number of CD4⁺ T cells and CD8⁺ T cells (CD4⁺/Treg Ratio and CD8⁺/Treg Ratio) compared to regulatory T cells (Treg cells) that regulate the immune system, indicate that CD4⁺ and CD8⁺ T cells increased more than the increase of Treg cells compared to the baseline, in all dose groups. See FIGS. 10A, 10B, and 10C.

4.4 Analysis of Changes in Ki67 Expression and ALC for Optimal Administration Interval Setting

4.4.A. Results of Preclinical Studies to Establish Dosing Interval

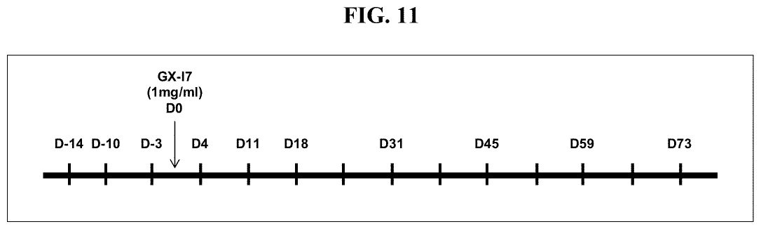

To confirm the reactivity of IL-7 fusion protein for setting the administration interval, Ki67 reactivity was confirmed 7 days after stimulation of IL-7 fusion protein at each time point in vitro using blood samples obtained in monkey experiments. Its blood sampling time point is shown in FIG. 11.

As a result, in PBMCs 14, 10, and 3 days before IL-7 fusion protein administration, the expression of Ki67 was increased by stimulation of IL-7 fusion protein, whereas after administration of IL-7 fusion protein. In blood samples obtained at the 4th, 11th, and 18th days, the expression of Ki67 by the IL-7 fusion protein was confirmed to decrease. This trend was observed in both CD4⁺ and CD8⁺ T cells, and it was confirmed that Ki67 expression was increased in blood samples at the 31st, 45th, 59th, and 73rd days.

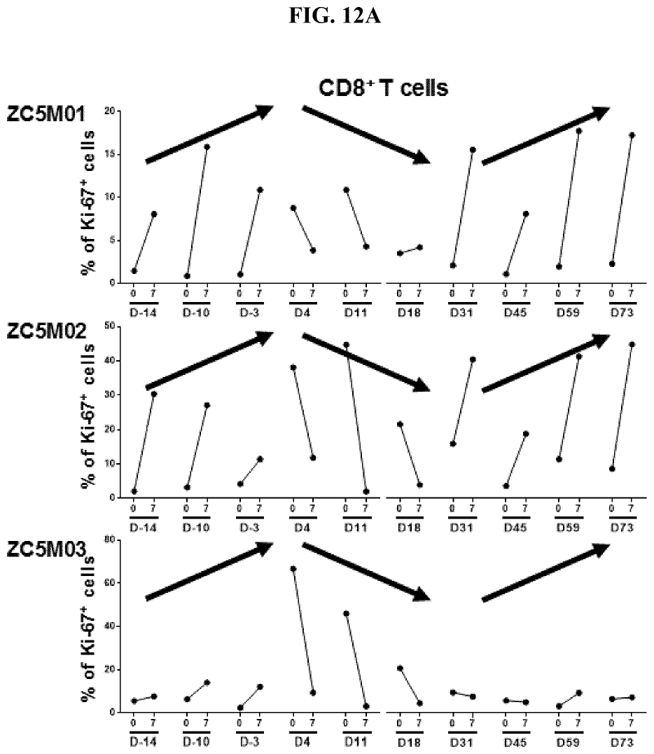

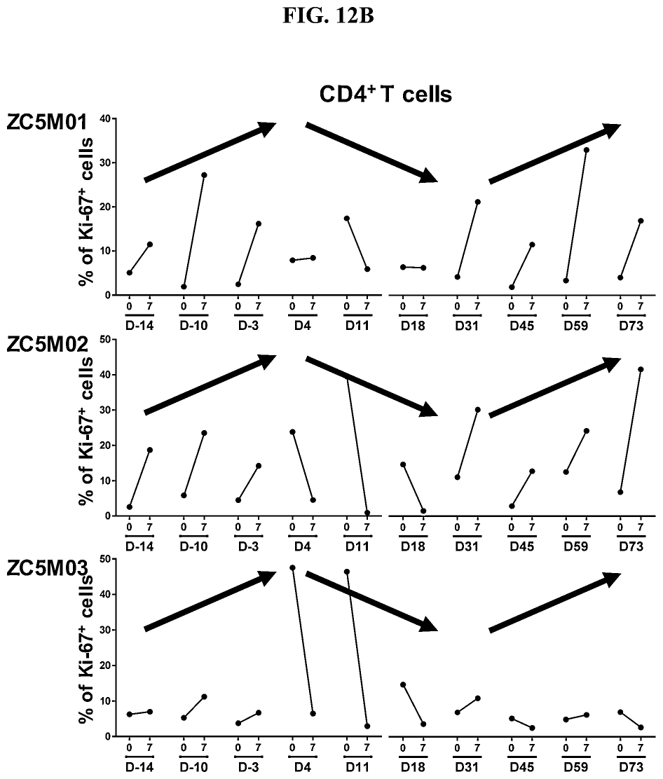

The results suggest that after the first proliferation of T cells by the IL-7 fusion protein, it may take a certain period of time before reproliferation appears. It is estimated that there will be an increasing inflection point. In addition, some individuals (ZC5M03) were observed to recover their responsiveness at about 5-6 weeks. FIGS. 12A and 12B.

4.4.B. Clinical Study Results for Setting Administration Interval

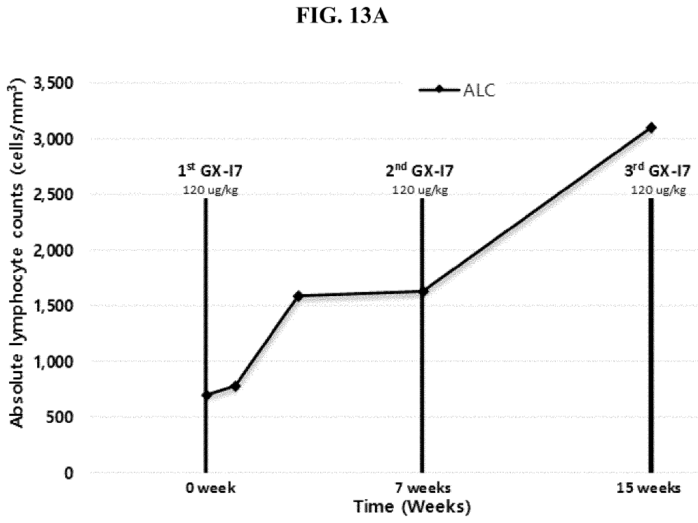

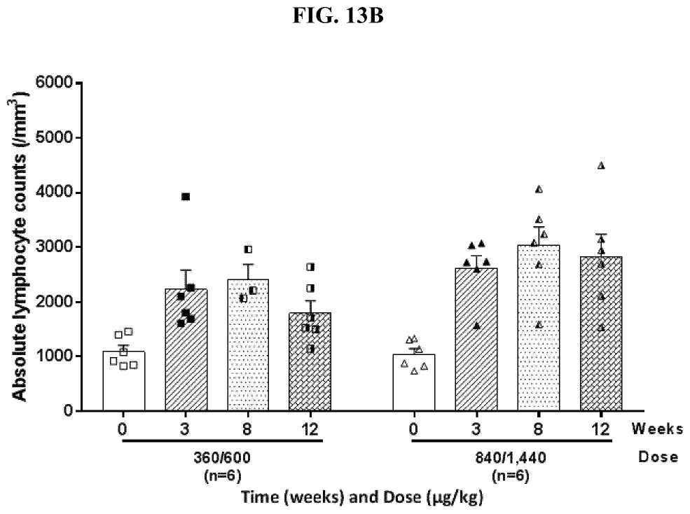

Results of measuring the changes in ALC according to single administration of IL-7 fusion protein as a single therapy in cancer patients showed that the ALC increased by the first administration in the high-dose group was maintained for up to 12 weeks. This suggests that it is possible to maintain an interval of 8 to 12 weeks or longer (e.g., 15 weeks) in setting the administration interval. See FIGS. 13A and 13B.

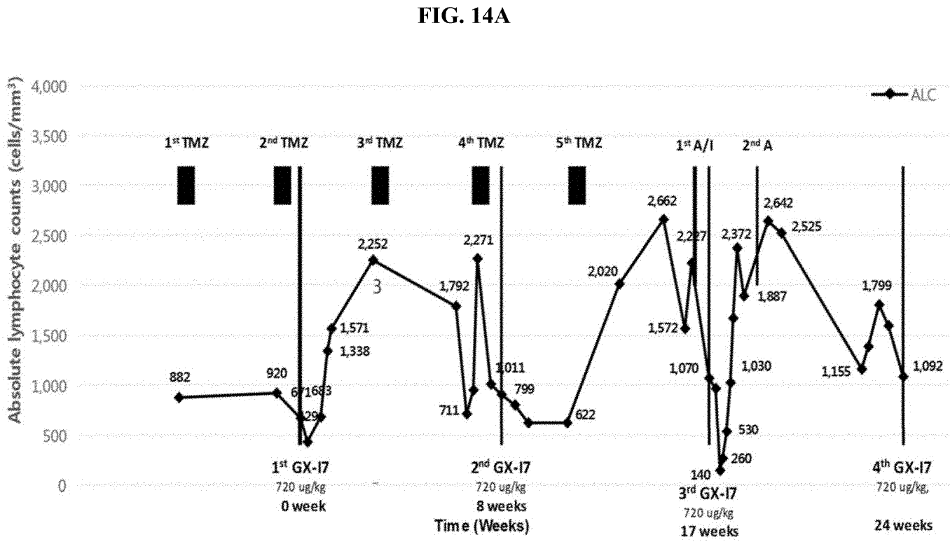

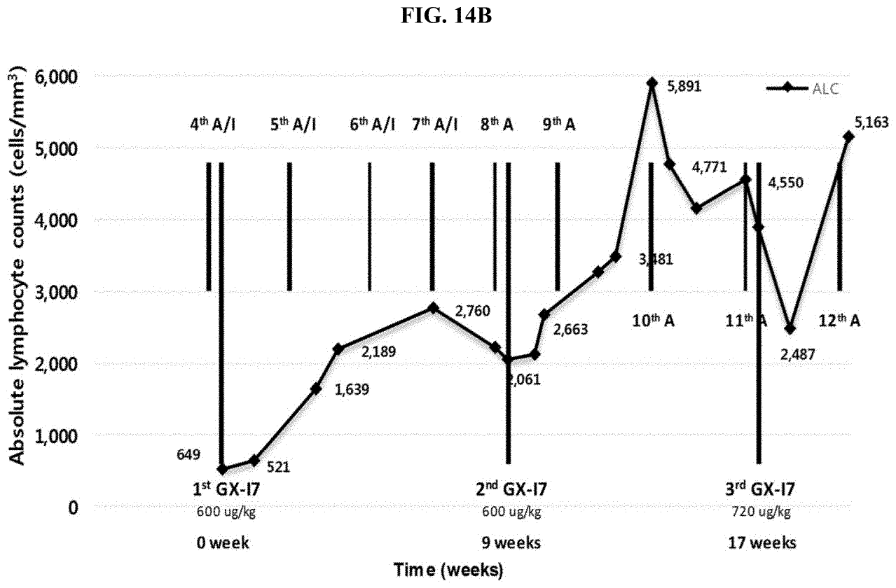

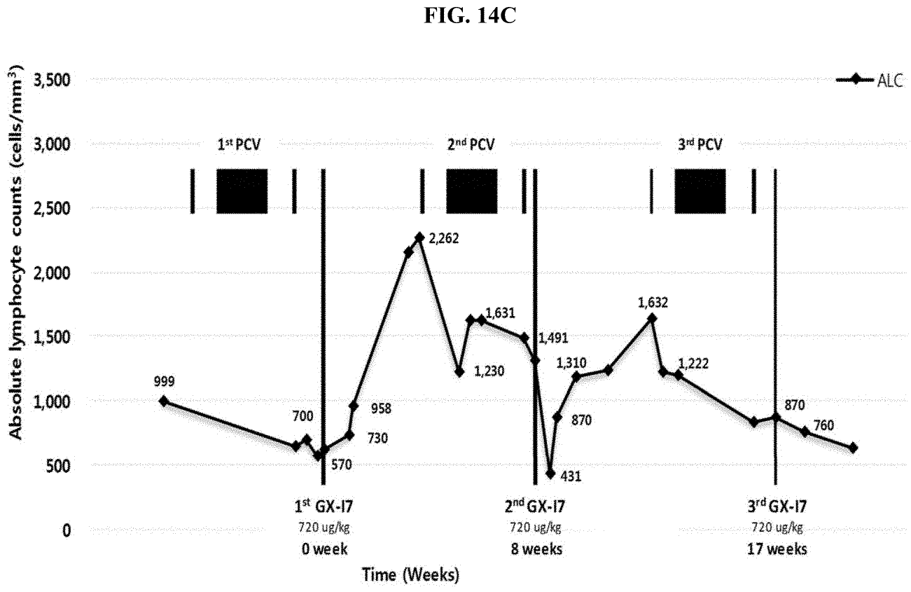

In a study on glioblastoma patients, the actual IL-7 fusion protein was not only administered alone, but also in combination with various other anticancer drugs, including chemotherapy (TMZ), as a standard treatment for glioblastoma patients. ALC was significantly increased by repeated administrations. The results show that the number of absolute lymphocyte count (ALC), which is usually reduced by existing chemotherapy drugs (that preferentially kill rapidly proliferating cells) can be maintained above a certain level by a single or repeated administration of IL-7 fusion protein. Therefore, the IL-7 fusion protein is expected to enhance or improve existing anticancer treatments. FIGS. 14A, 14B, and 14C.

4.5 Changes in T Cell Subsets, NK Cells, and B Cells Following Administration of IL-7 Fusion Protein

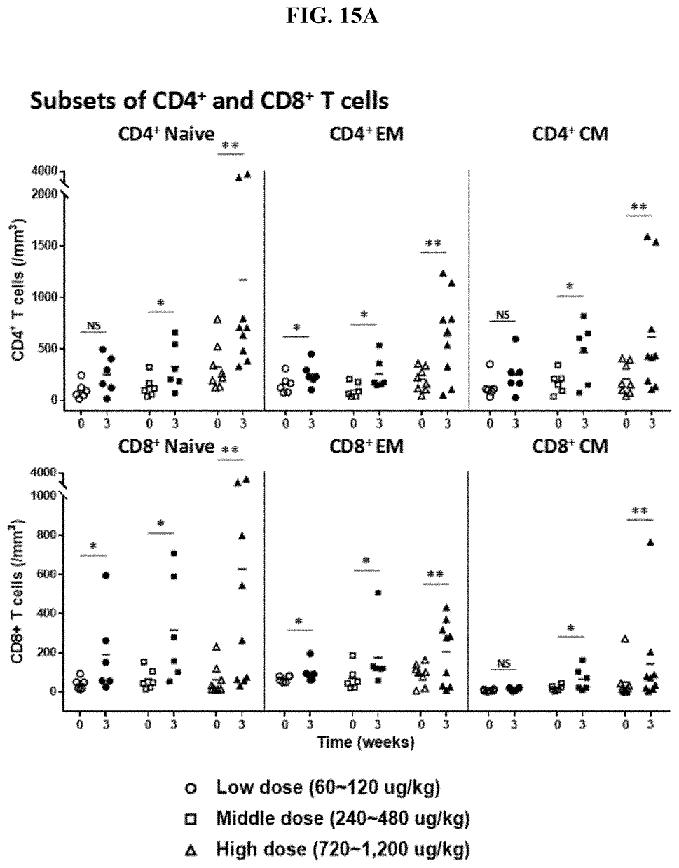

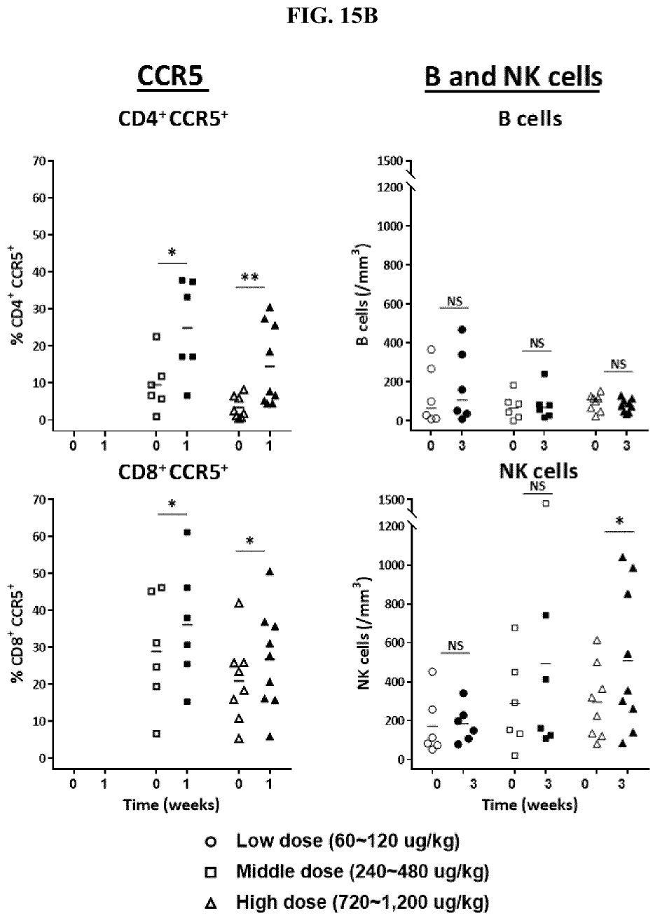

As a result of a subtype analysis of CD4⁺ T cells and CD8⁺ T cells in the patient group receiving GX-I7 as an IL-7 fusion protein, a dose-dependent increase of CD4⁺ T cells and CD8⁺ T cells was observed. The increases of Naïve CD4⁺ and CD8⁺ T cells were the largest compared to the baseline. Their CCR5 expression was also increased compared to the baseline value at each dose, suggesting that the IL-7 fusion protein administration could induce migration of T cells to the tumor site. NK cells also showed a dose-dependent increase compared to the baseline. However, no increase in B cells compared to the baseline was observed. See FIGS. 15A and 15B.

1. A method for increasing a lymphocyte count in a subject in need thereof, comprising administering
(i) a modified interleukin-7 of the following formula (I):
A−IL-7 formula (I)
wherein A is an oligopeptide consisting of 1 to 10 amino acid residues, and
the IL-7 is a polypeptide which is capable of binding to IL-7 receptor; or
(ii) an interleukin-7 fusion protein comprising
(a) the modified interleukin-7,
(b) a second domain comprising an oligopeptide having 1 to 10 amino acid residues consisting of methionine, glycine, or a combination thereof; and
(c) a third domain which prolongs the half-life of the interleukin-7 fusion protein, to the subject at a dose of greater than about 600 μg/kg.

2. The method of claim 1, wherein the subject is suffering from a cancer; infection; chronic failure of the right ventricle of the heart; Hodgkin's disease; a leak or rupture in the thoracic duct; side effects of prescription medications including anticancer agents (e.g., chemotherapy), antiviral agents, or glucocorticoids; malnutrition resulting from diets that are low in protein, radiation therapy, uremia, autoimmune disorders, immune deficiency syndromes, thymectomy, or a combination thereof, or idiopathic, acute radiation syndrome (ARS) or a combination thereof.

3. The method of claim 1 or 2, the IL-7 has an amino acid sequence selected from the group consisting of SEQ ID NOS: 1 to 6.

4. The method of claim 3, wherein the A is linked to the N-terminal of the IL-7.

5. The method of claim 3, wherein A is methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, or glycine-glycine-glycine.

6. The method of claim 5, wherein the third domain is linked to the N-terminal or C-terminal of the first domain or the second domain.

7. The method of any one of claims 4-6, wherein the third domain is any one selected from the group consisting of an Fc region of immunoglobulin or a part thereof, albumin, an albumin-binding polypeptide, Pro/Ala/Ser (PAS), a C-terminal peptide (CTP) of the 3 subunit of human chorionic gonadotropin, polyethylene glycol (PEG), long unstructured hydrophilic sequences of amino acids (XTEN), hydroxyethyl starch (HES), an albumin-binding small molecule, and a combination thereof.

8. The method of claim 7, wherein the third domain comprises an Fc region of a modified immunoglobulin.

9. The method of claim 8, wherein the modified immunoglobulin is selected from the group consisting of IgG1, IgG2, IgG3, IgG4, IgA1, IgA2, IgD, IgE and a combination thereof.

10. The method of claim 9, wherein the Fc region of the modified immunoglobulin comprises a hinge region, a CH2 domain, and a CH3 domain from the N-terminal to the C-terminal direction,
wherein the hinge region comprises a human IgD hinge region,
the CH2 domain comprises a part of the amino acid residues of CH2 domain of human IgD and human IgG4, and
the CH3 domain comprises a part of the amino acid residues of the human IgG4 CH3 domain.

11. The method of claim 10, wherein the Fc region of the modified immunoglobulin is represented by the following Formula (I):
N′—(Z1)p-Y—Z2-Z3-Z4-C′ Formula (I)
wherein N′ is the N-terminal of a polypeptide and C′ is the C-terminal of a polypeptide;
p is an integer of 0 or 1;
Z1 is an amino acid sequence having 5 to 9 consecutive amino acid residues from the amino acid residue at position 98 toward the N-terminal, among the amino acid residues at positions from 90 to 98 of SEQ ID NO: 7;
Y is an amino acid sequence having 5 to 64 consecutive amino acid residues from the amino acid residue at position 162 toward the N-terminal, among the amino acid residues at positions from 99 to 162 of SEQ ID NO: 7;
Z2 is an amino acid sequence having 4 to 37 consecutive amino acid residues from the amino acid residue at position 163 toward the C-terminal, among the amino acid residues at positions from 163 to 199 of SEQ ID NO: 7;
Z3 is an amino acid sequence having 71 to 106 consecutive amino acid residues from the amino acid residue at position 220 toward the N-terminal, among the amino acid residues at positions from 115 to 220 of SEQ ID NO: 8; and
Z4 is an amino acid sequence having 80 to 107 consecutive amino acid residues from the amino acid residue at position 221 toward the C-terminal, among the amino acid residues at positions from 221 to 327 of SEQ ID NO: 8.

12. The method of claim 1, wherein the third domain has an amino acid sequence selected from the group consisting of SEQ ID NOS: 9 to 14.

13. The method of claim 2, wherein the cancer is a solid tumor, a cancer of lymphatic system, or leukemia.

14. The method of claim 13, wherein the solid tumor is synovial sarcoma, infiltrating duct carcinoma, rectal cancer, colon cancer, ovary cancer, ascending colon cancer, anal cancer, invasive ductal carcinoma, adenocarcinoma, rectal cancer with paraaortic in metastatis, neuroendocrine carcinoma (cervix), sigmoid colon cancer, or glioblastoma.

15. The method of any one of claims 1, 2, 13, or 14, wherein the subject has previously received, concurrently receives, or will receive one or more of cancer treatments including surgery, radiation, and/or chemotherapy.

16. The method of claims 1 or 2, wherein the (i) modified interleukin-7 or the (ii) interleukin-7 fusion protein is administered at a dose in a range from greater than about 600 μg/kg to about 2,000 μg/kg.

17. The method of claims 1 or 2, wherein the (i) modified interleukin-7 or the (ii) interleukin-7 fusion protein is administered at a dose of about 720 μg/kg or above, about 960 μg/kg or above, about μg/kg or above, 1,200 μg/kg or above, about 1,700 μg/kg or above, or about 2,000 g/kg.

18. The method of claims 1 or 2, wherein the (i) modified interleukin-7 or the (ii) interleukin-7 fusion protein is administered at a dose at a dose of about 720 μg/kg or above, about 840 μg/kg or above, or about 1,440 μg/kg or above.

19. The method of claims 1 or 2, wherein the (i) modified interleukin-7 or the (ii) interleukin-7 fusion protein is administered at a dose of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg.

20. The method of claims 1 or 2, wherein the (i) modified interleukin-7 or the (ii) interleukin-7 fusion protein is administered twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks.

21. The method of claims 1 or 2, wherein the (i) modified interleukin-7 or the (ii) interleukin-7 fusion protein is administered twice or more times at an interval of 10 days, 20 days, 30 days, 40 days, 50 days, 60 days, 70 days, 80 days, 90 days, or 100 days.

22. The method of claims 1 or 2, wherein the (i) modified interleukin-7 or the (ii) interleukin-7 fusion protein is administered parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

23. The method of claims 1 or 2, comprising administering the (ii) interleukin-7 fusion protein.

24. The method of claim 23, wherein the (ii) interleukin-7 fusion protein comprises the amino acid sequence of SEQ ID NO: 24.

25. The method of claims 1 or 2, wherein the subject has a lymphocyte count of about 1000 lymphocyte cells or less/μl of blood, as determined according to Common Terminology Criteria for Adverse Events (CTCAE) version 4.0.

26. The method of claim 25, wherein the lymphocyte is T-cell.

27. The method of claims 1 or 2, wherein the (i) modified interleukin-7 or the (ii) interleukin-7 fusion protein is administered twice or more times in an amount of about 720 μg/kg at an interval of about 1 week, about 2 weeks, about 3 weeks, about 4 weeks, about 5 week, or about 6 weeks.

28. The method of claims 1 or 2, wherein the (i) modified interleukin-7 or the (ii) interleukin-7 fusion protein is administered twice or more times in an amount of about 840 μg/kg at an interval of about 2 weeks, about 3 weeks, about 4 weeks, about 5 weeks, or about 6 weeks.

29. The method of claims 1 or 2, wherein the (i) modified interleukin-7 or the (ii) interleukin-7 fusion protein is administered twice or more times in an amount of about 960 μg/kg at an interval of about 2 weeks, about 3 weeks, about 4 weeks, about 5 weeks, about 6 weeks, about 7 weeks, about 8 weeks, or about 9 weeks.

30. The method of claims 1 or 2, wherein the (i) modified interleukin-7 or the (ii) interleukin-7 fusion protein is administered twice or more times in an amount of about 1,200 μg/kg at an interval of about 3 weeks, about 4 weeks, about 5 weeks, about 6 weeks, about 7 weeks, about 8 weeks, about 9 weeks, or about 10 weeks.

31. The method of claims 1 or 2, wherein the (i) modified interleukin-7 or the (ii) interleukin-7 fusion protein is administered twice or more times in an amount of about 1,440 μg/kg at an interval of about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 2 months, or about 3 moths.

32. The method of claims 1 or 2, wherein the (i) modified interleukin-7 or the (ii) interleukin-7 fusion protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

33. The method of claims 1 or 2, wherein the (i) modified interleukin-7 or the (ii) interleukin-7 fusion protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

34. The method of claims 1 or 2, wherein the (i) modified interleukin-7 or the (ii) interleukin-7 fusion protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

35. The method of claims 1 or 2, wherein the (i) modified interleukin-7 or the (ii) interleukin-7 fusion protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

36. The method of claims 1 or 2, wherein the (i) modified interleukin-7 or the (ii) interleukin-7 fusion protein is administered at a dosing frequency of once a week, once in every two weeks, once in every three weeks, once in every four weeks, once in every five weeks, once in every six weeks, once in every seven weeks, once in every eight weeks, once in every nine weeks, once in every 10 weeks, once in every 11 weeks, once in every 12 weeks, once in every 13 weeks, once in every 14 weeks, or once in every 15 weeks.

37. The method of claim 26, wherein the T-cell is CD4⁺ and/or CD8⁺ T-cell.

38. The method of claim 26, wherein the T-cell is CD4⁺/CD8⁺ T-cell.

39. The method of claim 25, wherein the subject has a lymphocyte count of about 800 lymphocyte cells or less/μl of blood.

40. The method of claim 25, wherein the subject has a lymphocyte count of about 500 lymphocyte cells or less/μl of blood.

41. The method of claim 25, wherein the subject has a lymphocyte count of about 200 lymphocyte cells or less/μl of blood.

42. The method of claims 1 or 2, wherein the subject has been, is concurrently, or will be administered with an anti-cancer agent.

43. The method of claim 42, wherein the anti-cancer agent is an anti-cancer chemical compound.

44. The method of claims 25 or 42, wherein a number of tumor infiltrating lymphocytes (TILs) in the tumor is increased after the administration of the (i) modified interleukin-7 or the (ii) interleukin-7 fusion protein compared to a number of TILs in a tumor before the administration.

45. The method of claim 44, wherein the TILs are CD4⁺ TILs.

46. The method of claim 44, wherein the TILs are CD8⁺ TILs.

47. The method of claim 44, wherein the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

48. A use of the following (i) and/or (ii) for increasing a lymphocyte count in a subject in need thereof, comprising administering
(i) a modified interleukin-7 of the following formula (I):
A−IL-7 formula (I)
wherein A is an oligopeptide consisting of 1 to 10 amino acid residues, and
the IL-7 is a polypeptide which is capable of binding to IL-7 receptor; and/or
(ii) an interleukin-7 fusion protein comprising
(a) the modified interleukin-7,
(b) a second domain comprising an oligopeptide having 1 to 10 amino acid residues consisting of methionine, glycine, or a combination thereof; and
(c) a third domain which prolongs the half-life of the interleukin-7 fusion protein, to the subject at a dose of greater than about 600 μg/kg.

49. A use of the following (i) and/or (ii) in manufacturing a medicament for use in increasing a lymphocyte count in a subject in need thereof, said medicament being administered to the patient at a dose of greater than about 600 μg/kg,
(i) a modified interleukin-7 of the following formula (I):
A−IL-7 formula (I)
wherein A is an oligopeptide consisting of 1 to 10 amino acid residues, and
the IL-7 is a polypeptide which is capable of binding to IL-7 receptor; and/or
(ii) an interleukin-7 fusion protein comprising
(a) the modified interleukin-7,
(b) a second domain comprising an oligopeptide having 1 to 10 amino acid residues consisting of methionine, glycine, or a combination thereof; and
(c) a third domain which prolongs the half-life of the interleukin-7 fusion protein.

50. A pharmaceutical composition for increasing a lymphocyte count in a subject in need thereof, comprising as an active ingredient,
(i) a modified interleukin-7 of the following formula (I):
A−IL-7 formula (I)
wherein A is an oligopeptide consisting of 1 to 10 amino acid residues, and
the IL-7 is a polypeptide which is capable of binding to IL-7 receptor; and/or
(ii) an interleukin-7 fusion protein comprising
(a) the modified interleukin-7,
(b) a second domain comprising an oligopeptide having 1 to 10 amino acid residues consisting of methionine, glycine, or a combination thereof; and
(c) a third domain which prolongs the half-life of the interleukin-7 fusion protein, wherein said pharmaceutical composition is administered to the subject at a dose of greater than about 600 μg/kg.


SEQUENCE LISTING (40 sequences)

SEQ ID NO: 1 - protein, 177 aa, Homo sapiens
  amino acid sequence of human IL-7 (Accession number P13232)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
  amino acid sequence of rat IL-7 (Accession number P56478)
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus musculus
  amino acid sequence of mouse IL-7 (Accession number P10168)
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Chlorocebus sabaeus
  amino acid sequence of monkey IL-7 (Accession number  NP_001279008)
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
  amino acid sequence of cow IL-7 (Accession number P26895)
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
  amino acid sequence of sheep IL-7 (Accession number  Q28540)
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Homo sapiens
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Homo sapiens
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  Synthetic oligopeptide
MMMM